(19) United States
(12) Patent Application Publication
(10) Pub. No.: US 2023/0310324 A1
(43) Pub. Date: Oct. 5, 2023

(54) METHOD OF MAKING LYOPHILIZED PROTEIN FORMULATIONS

(71) Applicant: AMGEN INC., Thousand Oaks, CA (US)

(72) Inventors: Arnold McAuley, Thousand Oaks, CA (US); Michael J. Treuheit, Newbury Park, CA (US); Twinkle R. Christian, Thousand Oaks, CA (US); Bharadwaj Jagannathan, Thousand Oaks, CA (US)

(21) Appl. No.: 18/024,893

(22) PCT Filed: Sep. 14, 2021
(86) PCT No.: PCT/US21/50238

§ 371 Date: Mar. 6, 2023

Related U.S. Application Data
(60) Provisional application No. 63/077,908, filed on Sep. 14, 2020.

(51) Int. Cl. A61K 9/19; A61K 47/26; A61K 47/18; C07K 16/28

(52) U.S. Cl. CPC A61K 9/19; A61K 47/26; A61K 47/183; C07K 16/2863; C07K 2317/31

(57) ABSTRACT

Disclosed herein are methods of preparing lyophilized formulations comprising a protein, such as an antibody or a bispecific antibody construct, that exhibits improved storage stability.

### CROSS REFERENCE TO RELATED APPLICATIONS

This application claims the benefit of priority to U.S. Provisional Patent Application No. 63/077,908, filed Sep. 14, 2020, the disclosure of which is hereby incorporated by reference in its entirety.

### FIELD OF THE INVENTION

The disclosure provides methods for preparing lyophilized formulations comprising a protein, such as an antibody or bispecific antibody construct, that exhibits improved storage stability.

### INCORPORATION BY REFERENCE

Incorporated by reference in its entirety is a computer-readable nucleotide/amino acid sequence listing submitted concurrently herewith and identified as follows: ASCII (text) file named “55423_Seqlisting.txt”, 345,229 bytes created Sep. 14, 2020.

### BACKGROUND

Protein-based pharmaceuticals, such as pharmaceuticals that contain antibodies, antibody fragments, and bispecific antibody constructs, are becoming increasingly important for the treatment of various diseases and conditions. Proteins, however, are only marginally stable and are highly susceptible to both chemical and physical degradation. Chemical degradation refers to modifications involving covalent bonds, such as deamidation, oxidation, cleavage, clipping/fragmentation, formation of new disulfide bridges, hydrolysis, isomerization, or deglycosylation. Physical degradation includes protein unfolding, undesirable adsorption to surfaces, and aggregation. Dealing with these physical and chemical instabilities is one of the most challenging tasks in the development of protein pharmaceuticals (Chi et al., Pharm Res, Vol. 20, No. 9, September 2003, pp. 1325-1336, Roberts, Trends Biotechnol. 2014 July; 32(7):372-80).

Half-life extended antibody constructs (e.g., bispecific T cell engagers (BiTE®) comprising a half-life extending modality such as Fc-molecules), in particular, need to be protected against protein aggregation and/or other degradation events. Protein aggregation of BiTE® molecules is problematic because it can impair biological activity and quality (specifications) of the therapeutic proteins. Moreover, aggregation of BiTE® molecules may decrease product yield due to elaborate purification steps that are required to remove the aggregates from the end product. More recently, there has also been growing concern and evidence that the presence of aggregated proteins (even humanized or fully human proteins) can significantly increase the risk that a patient will develop an immune response to the active protein monomer, resulting in the formation of neutralizing antibodies and drug resistance, or other adverse side effects (Mahler J Pharm Sci. 2009 September; 98(9):2909-34).

Protein-based pharmaceutical formulations are often lyophilized and stored in the solid state to help preserve the integrity of the protein, such as the antibody or bispecific antibody construct, in the formulation during storage. Many current methods of lyophilizing protein formulations, however, fail to result in solid state formulations that exhibit suitable stability over time. Thus, there is a need for new methods of producing lyophilized protein formulations that exhibit improved storage stability.

### SUMMARY

In one aspect, the disclosure provides a method of preparing a lyophilized formulation, the method including (a) cooling a lyophilization chamber containing a liquid formulation comprising a protein, a saccharide, and a surfactant to a temperature ranging from about −35° C. to about −50° C. to produce a frozen formulation, and holding the chamber at a temperature ranging from about −40° C. to about −50° C. for a time period of about 2 hours to about 24 hours; (b) heating the chamber to a temperature ranging from about −30° C. to about −20° C. and a pressure ranging from about 25 mTorr to about 100 mTorr to produce a primary dried formulation, and holding the chamber at a temperature ranging from about −30° C. to about −20° C. and a pressure ranging from about 25 mTorr to about 100 mTorr for a time period of about 45 hours to about 60 hours; (c) heating the chamber to a temperature ranging from about 20° C. to about 35° C. to produce a secondary dried formulation, and holding the chamber at a temperature ranging from about 20° C. to about 30° C. and a pressure ranging from about 25 mTorr to about 100 mTorr for a time period of about 5 hours to about 10 hours to produce the lyophilized formulation; wherein the liquid formulation has a pH or about 3-7 and does not contain mannitol; and the method lacks an annealing step.

In another aspect, the disclosure provides a lyophilized protein formulation prepared by the method of the disclosure.

Further aspects and advantages will be apparent to those of ordinary skill in the art from a review of the following detailed description. While the methods disclosed herein are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

FIG. 1 shows a comparison of clipping levels measured by rCE-SDS after 1 month of storage at 25° C. for liquid and reconstituted lyophilized formulations (that were prepared with an annealing step) containing 1 mg/mL of a bispecific antibody construct comprising a sequence set forth in SEQ ID NO: 22 (BiTE A), SEQ ID NO: 77 (BiTE B), SEQ ID NO: 87 (BiTE C), and SEQ ID NO: 97 (BiTE D). The reconstituted lyophilized formulation shows no significant increase in clipping lost under accelerated stress conditions.

FIG. 2 shows a comparison of clipping levels measured by rCE-SDS after 1 month of storage at 40° C. for liquid and reconstituted lyophilized (that were prepared with an annealing step) formulations of BiTE B at 1 mg/mL.

FIG. 3 shows the percentage high molecular weight (HMW) species measured by SE-UHPLC for a reconstituted lyophilized formulation (that was prepared with an annealing step) containing BiTE B at varying protein concentrations after 1 month of storage at 40° C. There is no increase in % HMW under accelerated stress conditions, demonstrating the applicability of the lyophilization cycle for high concentration protein formulations, such as high concentration antibody or bispecific antibody construct formulations.

FIG. 4 shows an increase in % HMW measured by SE-UHPLC after storing a lyophilized formulation containing 23 mg/mL of BiTE B at the frozen temperatures experienced by the protein during the lyophilization cycle. The ‘annealed’ sample was stored at −45° C. for 48 hours followed by −12° C. storage for 5 hours, −45° C. storage for 5 hours and −25° C. storage for 48 hours. The ‘non-annealed’ sample was stored at −45° C. for 58 hours followed by −25° C. storage for 48 hours.

FIG. 5 shows the lyophilization cycle (using no annealing step) for a placebo formulation containing 10 mM glutamic acid, 9% (w/v) sucrose, and 0.01% (w/v) polysorbate 80) with a drying temperature of −10° C., which did not induce a cake collapse. Cake integrity was acceptable. Some curvature was observed in the cakes.

### DETAILED DESCRIPTION

Disclosed herein are methods of preparing lyophilized formulations comprising a protein, such as an antibody or a bispecific antibody construct (e.g., a half-life extended bispecific antibody construct), which exhibit improved stability. The lyophilization methods of the disclosure advantageously result in decreased physical degradation, such as aggregation, as well as decreased chemical degradation, such as decreased clipping and deamidation. Furthermore, the lyophilization methods disclosed herein are able to stabilize both low and high concentration protein formulations, such as formulations containing antibodies and bispecific antibody constructs.

### Definitions

As used herein, the term “pharmaceutical formulation” relates to a formulation which is suitable for administration to a subject in need thereof. The terms “subject” or “individual” or “animal” or “patient” are used interchangeably herein to refer to any subject, particularly a mammalian subject, for whom administration of the pharmaceutical formulation of the disclosure is desired. Mammalian subjects include humans, non-human primates, dogs, cats, guinea pigs, rabbits, rats, mice, horses, cattle, cows, and the like, with humans being preferred. The pharmaceutical formulation of the present disclosure is stable and pharmaceutically acceptable, i.e., capable of eliciting the desired therapeutic effect without causing significant undesirable local or systemic effects in the subject to which the pharmaceutical formulation is administered. Pharmaceutically acceptable formulations of the disclosure may be sterile. Specifically, the term “pharmaceutically acceptable” can mean approved by a regulatory agency or other generally recognized pharmacopoeia for use in animals, and more particularly in humans, but is not limited to those approved by a regulatory agency.

The term “stability” or “stabilization” relates to the stability of the pharmaceutical formulation in total and in particular to the stability of the active ingredient (e.g. the protein, such as a bispecific antibody construct) itself, specifically during formulation, filling, shipment, storage and administration. A “stable formulation” is one in which the protein (e.g., an antibody or bispecific antibody construct) therein essentially retains its physical and/or chemical integrity and biological activity upon storage and during processes (such as freeze/thaw, mechanical mixing and lyophilization). Protein stability can be measured by formation of high molecular weight (HMW) species, loss of enzyme activity, generation of peptide fragments and shift of charge profiles, as described in the Stability of Lyophilized Protein Formulation section infra.

The term “aggregation” as used herein refers to the direct mutual attraction between molecules, e.g. via van der Waals forces or chemical bonding. In particular, aggregation is understood as proteins accumulating and clumping together. Aggregates may include amorphous aggregates and oligomers, and are typically referred to as high molecular weight (HMW) species, i.e. molecules having a higher molecular weight than product molecules which are non-aggregated molecules.

The term “(protein) aggregate” as used herein generally encompasses protein species of higher molecular weight such as “oligomers” or “multimers” instead of the desired defined species (e.g., a monomer). The term is used interchangeably herein with the terms “high molecular weight” species and “HMW”. Protein aggregates may generally differ in size (ranging from small (dimers) to large assemblies (subvisible or even visible particles) and from the nanometer to micrometer range in diameter), morphology (approximately spherical to fibrillar), protein structure (native vs. non-native/denatured), type of intermolecular bonding (covalent vs. non-covalent), reversibility and solubility. Soluble aggregates cover the size range of roughly 1 to 100 nm, and protein particulates cover subvisible (˜0.1-100 nm) and visible (>100 nm) ranges. All of the aforementioned types of protein aggregates are generally encompassed by the term. The term “(protein) aggregate” thus refers to all kinds physically-associated or chemically linked non-native species of two or more protein monomers.

The term “low molecular weight (LMW)” species as used herein refers to fragments of a protein, such as a bispecific antibody construct.

### Methods

One aspect of the disclosure provides a method of preparing a lyophilized formulation, wherein the method lacks an annealing step. The method comprises: (a) cooling a lyophilization chamber containing a liquid formulation having a pH of about 3-7 and comprising a protein, a saccharide, and a surfactant, and lacking mannitol, to a temperature ranging from about −35° C. to about −50° C. to produce a frozen formulation, and holding the chamber at a temperature ranging from about −40° C. to about −50° C. for a time period of about 2 hours to about 24 hours; (b) heating the chamber to a temperature ranging from about −30° C. to about −20° C. and a pressure ranging from about 25 mTorr to about 100 mTorr to produce a primary dried formulation, and holding the chamber at a temperature ranging from about −30° C. to about −20° C. and a pressure ranging from about 25 mTorr to about 100 mTorr for a time period of about 45 hours to about 60 hours; and (c) heating the chamber to a temperature ranging from about 20° C. to about 35° C. to produce a secondary dried formulation, and holding the chamber at a temperature ranging from about 20° C. to about 30° C. and a pressure ranging from about 25 mTorr to about 100 mTorr for a time period of about 5 hours to about 10 hours to produce the lyophilized formulation. The term “temperature” as used herein refers to a temperature that is internal to the lyophilization chamber (i.e., the internal temperature of the lyophilization chamber “internal temperature”). Likewise, the term “pressure” as used herein refers to a pressure that is internal to the lyophilization chamber (i.e., the internal pressure of the lyophilization chamber “internal pressure”).

Step (a). In step (a), the lyophilization chamber containing the liquid formulation is cooled to a temperature (e.g., internal temperature) ranging from about −35° C. to about −50° C. to produce a frozen formulation, and held at a temperature (e.g., internal temperature) ranging from about −40° C. to about −50° C. for a time period of about 2 hours to about 24 hours. In some embodiments, the cooling occurs to a temperature ranging from about −40° C. to about −50° C. (e.g., about −40° C., −41° C., −42° C., −43° C., −44° C., −45° C., −46° C., −47° C., −48° C., −49° C., or −50° C.). In various cases, the cooling can occur to a temperature of about −45° C. In some cases, the cooling of the chamber occurs at a rate ranging from about 0.5° C./min to about 1° C./min. In various embodiments, the cooling occurs at a rate from about 0.5° C./min to about 0.8° C./min. In some embodiments, the cooling occurs at a rate of about 0.5° C./min, 0.6° C./min, 0.7° C./min, 0.8° C./min, 0.9° C./min, or 1° C./min. In some cases, the cooling occurs at a rate of about 0.5° C./min. In some embodiments, the holding of the chamber can occur at a temperature of about −40° C., −41° C., −42° C., −43° C., −44° C., −45° C., −46° C., −47° C., −48° C., −49° C., or −50° C. In some embodiments, the holding occurs at a temperature of about −45° C. In some embodiments, the temperature to which the lyophilization chamber is cooled and the holding temperature are the same. In various embodiments, the holding occurs for a time period of about 2 hours to about 5 hours (e.g., about 2 hours, 2.5 hours, 3 hours, 3.5 hours, 4 hours, 4.5 hours, or 5 hours). In some cases, the holding occurs for about 2 hours.

Step (b). In step (b), the lyophilization chamber is heated to a temperature (e.g., internal temperature) ranging from about −30° C. to about −20° C. and a pressure (e.g., internal pressure) ranging from about 25 mTorr to about 100 mTorr to produce a primary dried formulation, and held at a temperature (e.g., internal temperature) ranging from about −30° C. to about −20° C. and a pressure (e.g., internal pressure) ranging from about 25 mTorr to about 100 mTorr for a time period of about 45 hours to about 60 hours. In some embodiments, the heating occurs to a temperature of about −30° C., −29° C., −28° C., −27° C., −26° C., −25° C., −24° C., −23° C., −22° C., −21° C., or −20° C. In various cases, the heating occurs to a temperature of about −25° C. In some cases, the heating occurs at a rate ranging from about 0.1° C./min to about 1° C./min. In various embodiments, the heating occurs at a rate from about 0.1° C./min to about 0.5° C./min (e.g., 0.1° C./min, 0.2° C./min, 0.3° C./min, 0.4° C./min, or 0.5° C./min). In some cases, the heating occurs at a rate of about 0.3° C./min. In various cases, the heating occurs at a pressure ranging from about 25 mTorr to about 75 mTorr, or about 50 mTorr to about 100 mTorr, or about 70 mTorr to about 100 mTorr, or about 65 mTorr to about 75 mTorr. In some cases, the heating occurs at a pressure of about 65 mTorr, 66 mTorr, 67 mTorr, 68 mTorr, 69 mTorr, 70 mTorr, 71 mTorr, 72 mTorr, 73 mTorr, 74 mTorr, or 75 mTorr. In various embodiments, the heating occurs at a pressure of about 70 mTorr. In some embodiments, the holding of the chamber occurs at a temperature of about −30° C., −29° C., −28° C., −27° C., −26° C., −25° C., −24° C., −23° C., −22° C., −21° C., or −20° C. In various cases, the holding occurs at a temperature of about −25° C. In various embodiments, the holding occurs at a pressure ranging from about 25 mTorr to about 75 mTorr, or about 50 mTorr to about 100 mTorr, or about 70 mTorr to about 100 mTorr, or about 65 mTorr to about 75 mTorr. In some cases, the holding occurs at a pressure of about 65 mTorr, 66 mTorr, 67 mTorr, 68 mTorr, 69 mTorr, 70 mTorr, 71 mTorr, 72 mTorr, 73 mTorr, 74 mTorr, or 75 mTorr. In various embodiments, the holding occurs at a pressure of about 70 mTorr. In some embodiments, the temperature to which the lyophilization chamber is heated and the holding temperature are the same. In some embodiments, the pressure under which the lyophilization chamber is heated and the holding pressure are the same. In some embodiments, the temperature under which the lyophilization chamber is heated and the holding temperature are the same, and also the pressure under which the lyophilization chamber is heated and the holding pressure are the same. In some cases, the holding occurs for a time period of about 50 hours to about 55 hours (e.g., about 50 hours, 51 hours, 52 hours, 53 hours, 54 hours, or 55 hours). In various cases, the holding occurs for a time period of about 52 hours.

Step (c). In step (c), the chamber is heated to a temperature (e.g., internal temperature) ranging from about 20° C. to about 35° C. to produce a secondary dried formulation, and held at a temperature (e.g., internal temperature) ranging from about 20° C. to about 30° C. and a pressure (e.g., internal pressure) ranging from about 25 mTorr to about 100 mTorr for a time period of about 5 hours to about 10 hours to produce the lyophilized formulation. In some embodiments, the heating occurs to a temperature of about 20° C., 21° C., 22° C., 23° C., 24° C., 25° C., 26° C., 27° C., 28° C., 29° C., 30° C., 31° C., 32° C., 33° C., 34° C., or 35° C. In various cases, the heating occurs to a temperature of about 30° C. In various embodiments, the heating occurs at a rate ranging up to about 0.5° C./min to produce the secondary dried formulation. In some cases, the heating occurs at a rate from about 0.05° C./min to about 0.5° C./min. In various cases, the heating occurs at a rate of about 0.05° C./min, 0.1° C./min, 0.15° C./min, 0.2° C./min, 0.25° C./min, 0.3° C./min, 0.35° C./min, 0.4° C./min, 0.45° C./min, or 0.5° C./min. In some embodiments, the heating occurs at a rate of about 0.1° C./min. In some embodiments, the holding occurs at a temperature of about 20° C., 21° C., 22° C., 23° C., 24° C., 25° C., 26° C., 27° C., 28° C., 29° C., or 30° C. In various cases, the holding occurs at a temperature of about 30° C. In some embodiments, the temperature to which the lyophilization chamber is heated is the same as the holding temperature. In some embodiments, the holding occurs at a pressure ranging from about 25 mTorr to about 75 mTorr, or about 50 mTorr to about 100 mTorr, or about 70 mTorr to about 100 mTorr, or about 65 mTorr to about 75 mTorr. In some cases, the holding occurs at a pressure of about 65 mTorr, 66 mTorr, 67 mTorr, 68 mTorr, 69 mTorr, 70 mTorr, 71 mTorr, 72 mTorr, 73 mTorr, 74 mTorr, or 75 mTorr. In various embodiments, the holding occurs at a pressure of about 70 mTorr. In some cases, the holding occurs for a time period of about 5 hours, 6 hours, 7 hours, 8 hours, 9 hours, or 10 hours. In various cases, the holding occurs for a time period of about 8 hours.

Another aspect of the disclosure provides a method of preparing a lyophilized formulation, wherein the method comprises an annealing step. The method of this aspect comprises: (a) cooling a lyophilization chamber containing a liquid formulation having a pH of about 3-7 and comprising a protein, a saccharide, and a surfactant, and lacking mannitol, to a temperature ranging from about −35° C. to about −50° C. to produce a frozen formulation, and holding the chamber at a temperature ranging from about −40° C. to about −50° C. for a time period of about 2 hours to about 24 hours; (b) heating the chamber to a temperature ranging from about −30° C. to about −20° C. and a pressure ranging from about 25 mTorr to about 100 mTorr to produce a primary dried formulation, and holding the chamber at a temperature ranging from about −30° C. to about −20° C. and a pressure ranging from about 25 mTorr to about 100 mTorr for a time period of about 45 hours to about 60 hours; and (c) heating the chamber to a temperature ranging from about 20° C. to about 35° C. to produce a secondary dried formulation, and holding the chamber at a temperature ranging from about 20° C. to about 30° C. and a pressure ranging from about 25 mTorr to about 100 mTorr for a time period of about 5 hours to about 10 hours to produce the lyophilized formulation, as previously described supra, but with an annealing step between steps (a) and (b). As used herein, “annealing” refers to a process in which the temperature of the formulation is cycled (e.g., from a low temperature to a higher temperature, and then back to the low temperature) to obtain more complete crystallization. In embodiments, the annealing step can include: (i) heating the chamber containing the frozen formulation from step (a) and holding the chamber at the heated temperature for a period of time; and (ii) cooling the chamber containing the frozen formulation back to the temperature of step (a) and holding the chamber containing the frozen formulation at a temperature ranging from about −35° C. to about −50° C. for a time period of about 2 hours to about 24 hours.

Step (i). In step (i), the heating of the frozen formulation from step (a) can occur to a temperature ranging from about −20° C. to about −5° C. at a rate ranging from about 0.1° C./min to about 1° C./min. In some embodiments, the heating occurs to a temperature of about −20° C., −19° C., −18° C., −17° C., −16° C., −15° C., −14° C., −13° C., −12° C., −11° C., −10° C., −9° C., −8° C., −7° C., −6° C., or −5° C. In some embodiments, the heating occurs to a temperature ranging from about −15° C. to about −10° C. In various cases, the heating occurs to a temperature of about −12° C. In various embodiments, the heating occurs at a rate from about 0.1° C./min to about 0.5° C./min (e.g., 0.1° C./min, 0.2° C./min, 0.3° C./min, 0.4° C./min, or 0.5° C./min). In some cases, the heating occurs at a rate of about 0.5° C./min. The holding of the frozen formulation can occur at a temperature ranging from about −20° C. to about −5° C. for a time period of about 2 hours to about 10 hours. In some embodiments, the holding occurs to a temperature of about −20° C., −19° C., −18° C., −17° C., −16° C., −15° C., −14° C., −13° C., −12° C., −11° C., −10° C., −9° C., −8° C., −7° C., −6° C., or −5° C. In some embodiments, the holding occurs at a temperature ranging from about −15° C. to about −10° C. In various cases, the holding occurs at a temperature of about −12° C. In various embodiments, the holding occurs for a time period of about 2 hours to about 5 hours (e.g., about 2 hours, 2.5 hours, 3 hours, 3.5 hours, 4 hours, 4.5 hours, or 5 hours). In some cases, the holding occurs for about 2 hours.

Step (ii). In step (ii), the cooling can occur to a temperature ranging from about −35° C. to about −50° C. at a rate ranging from about 0.5° C./min to about 1° C./min. In some embodiments, the cooling occurs to a temperature ranging from about −40° C. to about −50° C. (e.g., about −40° C., −41° C., −42° C., −43° C., −44° C., −45° C., −46° C., −47° C., −48° C., −49° C., or −50° C.). In various cases, the cooling occurs to a temperature of about −45° C. In various embodiments, the cooling occurs at a rate from about 0.5° C./min to about 0.8° C./min. In some embodiments, the cooling occurs at a rate of about 0.5° C./min, 0.6° C./min, 0.7° C./min, 0.8° C./min, 0.9° C./min, or 1° C./min. In some cases, the cooling occurs at a rate of about 0.5° C./min. In some embodiments, the holding can occur at a temperature ranging from about −40° C. to about −50° C. for a time period of about 2 hours to about 24 hours. In some embodiments, the holding occurs at a temperature of about −40° C., −41° C., −42° C., −43° C., −44° C., −45° C., −46° C., −47° C., −48° C., −49° C., or −50° C. In some embodiments, the holding occurs at a temperature of about −45° C. In various embodiments, the holding occurs for a time period of about 2 hours to about 5 hours (e.g., about 2 hours, 2.5 hours, 3 hours, 3.5 hours, 4 hours, 4.5 hours, or 5 hours). In some cases, the holding occurs for about 2 hours.

In embodiments of either method disclosed herein (with or without an annealing step), the method can further comprise step (d) cooling the chamber comprising the lyophilized formulation from step (c) to a temperature ranging from about 1° C. to about 10° C. (or to about 2° C. to about 7° C., or to about 5° C.) and aerating the lyophilized formulation with an inert gas at a pressure ranging from about 250 mTorr to about 750 mTorr (or to about 300 mTorr to about 600 mTorr, or to about 500 mTorr). In some cases, the inert gas is selected from argon, helium, nitrogen, and any combination thereof. In various cases, the inert gas is nitrogen. In embodiments, step (d) can facilitate stoppering of the container (e.g., a vial), which contains the lyophilized formulation. In embodiments, the method further comprises storing the lyophilized formulation at a temperature ranging from about 2° C. to about 8° C. In embodiments, the method further comprises reconstituting the lyophilized formulation with water.

In yet another aspect, the disclosure provides a lyophilized protein formulation prepared by a method disclosed herein. In some embodiments, the protein formulation is prepared by a method disclosed herein that lacks an annealing step. In various embodiments, the method disclosed herein comprises an annealing step.

### Lyophilized Protein Formulation

The lyophilized protein formulations described herein include a protein, a saccharide, a surfactant, and optionally a buffer, and have a pH of about 3 to about 7 (or about 3.5, 4, 4.5, 5, 5.5, 6, 6.5, or 7). In some cases, the pH is about 4 to about 6. In some cases the pH of the formulation is about 4, or about 4.2. In various cases, the pH of the formulation is about 5. In some embodiments, the pH of the formulation is about 6. In embodiments, the lyophilized formulation disclosed herein does not contain a sugar alcohol. As used herein, “sugar alcohol” refers to a linear polyol in which one hydroxyl group is attached to each carbon atom. Examples of sugar alcohols as used herein include xylitol, erythritol, mannitol, and sorbitol. In embodiments, the lyophilized formulation does not contain mannitol.

Protein

In some embodiments, the protein of the lyophilized formulation is an antigen-binding protein. An “antigen-binding protein” is a protein comprising a domain that binds a specified target antigen (such as CD3 and/or CDH19, MSLN, DLL3, FLT3, EGFRvIII, BCMA, PSMA, CD33, CD19, CD70, CLDN18.2 or MUC17). An antigen-binding protein comprises a scaffold or framework portion that allows the antigen binding domain to adopt a conformation that promotes binding of the antigen-binding protein to the antigen.

In some embodiments, the antigen-binding protein of the lyophilized formulation is an antibody or immunoglobulin, or an antigen-binding antibody fragment. In some cases, the antigen-binding protein is an antibody. The term “antibody” refers to an intact antigen-binding immunoglobulin. An “antibody” is a type of an antigen-binding protein. The antibody can be an IgA, IgD, IgE, IgG, or IgM antibody, including any one of IgG1, IgG2, IgG3 or IgG4. In various embodiments, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

In some embodiments, the antibody of the formulation is a bispecific antibody, i.e., an antibody that binds two different targets (e.g., CD3 and a second, different target). The term “bispecific” as used herein refers to an antibody construct that binds to two different target antigens, i.e., it comprises a first binding domain and a second binding domain, wherein the first binding domain binds to one antigen or target (e.g., the target cell surface antigen), and the second binding domain binds to another antigen or target (e.g. CD3). Accordingly, antibody constructs according to the disclosure comprise specificities for two different antigens or targets. The term “target cell surface antigen” refers to an antigenic structure expressed by a cell and which is present at the cell surface such that it is accessible for an antibody construct as described herein. The target cell surface antigen can be a protein, such as the extracellular portion of a protein, or a carbohydrate structure, such as a carbohydrate structure of a protein, such as a glycoprotein. The target cell surface antigen can be a tumor antigen. The disclosure also encompasses multispecific antibody constructs such as trispecific antibody constructs, the latter ones including three binding domains, or constructs having more than three (e.g. four, five, or more) specificities.

Bispecific antibodies and/or antibody constructs as understood herein include, but are not limited to, traditional bispecific immunoglobulins (e.g., BsIgG), IgG comprising an appended antigen-binding domain (e.g., the amino or carboxy termini of light or heavy chains are connected to additional antigen-binding domains, such as single domain antibodies or paired antibody variable domains (e.g., Fv or scFv)), BsAb fragments (e.g., bispecific single chain antibodies), bispecific fusion proteins (e.g., antigen binding domains fused to an effector moiety), and BsAb conjugates. See, e.g., Spiess et al., Molecular Immunology 67(2) Part A: 97-106 (2015), which describes various bispecific formats and is hereby incorporated by reference. Examples of bispecific constructs include, but are not limited to, diabodies, single chain diabodies, tandem scFvs, bispecific T cell engager (BiTE®) format (a fusion protein consisting of two single-chain variable fragments (scFvs) joined by a linker), and Fab2 bispecifics, as well as engineered constructs comprising full length antibodies. See, e.g., Chames & Baty, 2009, mAbs 1[6]:1-9; and Holliger & Hudson, 2005, Nature Biotechnology 23[9]:1126-1136; Wu et al., 2007, Nature Biotechnology 25[11]:1290-1297; Michaelson et al., 2009, mAbs 1[2]:128-141; International Patent Publication No. 2009032782 and 2006020258; Zuo et al., 2000, Protein Engineering 13[5]:361-367; U.S. Patent Application Publication No. 20020103345; Shen et al., 2006, J Biol Chem 281[16]:10706-10714; Lu et al., 2005, J Biol Chem 280[20]:19665-19672; and Kontermann, 2012 MAbs 4(2):182, all of which are expressly incorporated herein.

In some embodiments, the lyophilized formulations described herein comprise a bispecific antibody construct comprises a first binding domain that binds to a target cell surface antigen, a second binding domain that binds to human CD3 on the surface of a T cell, and optionally a third domain comprising, in an amino to carboxyl order, hinge-CH2 domain-CH3 domain-linker-hinge-CH2 domain-CH3 domain. In some embodiments, each of the first and second binding domains comprise a VH region and a VL region.

The term “binding domain” as used herein refers to a domain which (specifically) binds to/interacts with/recognizes a given target epitope or a given target site on the target molecules (antigens), e.g. CDH19, MSLN, DLL3, FLT3, EGFRvIII, BCMA, PSMA, CD33, CD19, CD70, CLDN18.2 or MUC17 and CD3, respectively.

The structure and function of the first binding domain (recognizing e.g. CDH19, MSLN, DLL3, FLT3, EGFRvIII, BCMA, PSMA, CD33, CD19, CD70, CLDN18.2 or MUC17) and also the structure and/or function of the second binding domain (recognizing CD3), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. In embodiments, the first binding domain is characterized by the presence of three light chain CDRs (i.e. CDR1, CDR2 and CDR3 of the VL region) and/or three heavy chain CDRs (i.e. CDR1, CDR2 and CDR3 of the VH region). In embodiments, the second binding domain also comprises the minimum structural requirements of an antibody which allow for the target binding. In embodiments, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

In some embodiments, the first binding domain which binds to the target cell surface antigen and/or the second binding domain which binds to CD3c is/are human binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. To avoid the use of rodent derived antibodies or antibody constructs, human or fully human antibodies/antibody constructs can be generated through the introduction of human antibody function into a rodent so that the rodent produces fully human antibodies.

In some embodiments, the antigen binding protein comprises a single chain antibody construct. A scFv comprises a variable heavy chain, a scFv linker, and a variable light domain. Optionally, the C-terminus of the variable light chain is attached to the N-terminus of the scFv linker, the C-terminus of which is attached to the N-terminus of a variable heavy chain (N-vh-linker-vl-C), although the configuration can be switched (N-vl-linker-vh-C). Alternatively, the C-terminus of the variable heavy chain is attached to the N-terminus of the scFv linker, the C-terminus of which is attached to the N-terminus of a variable light chain (N-vl-linker-vh-C), although the configuration can be switched (N-vh-linker-v-C). Thus, specifically included in the depiction and description of scFvs are the scFvs in either orientation.

The at least two binding domains and the variable domains (VH/VL) of the antibody construct of the present disclosure may or may not comprise peptide linkers (spacer peptides). The term “peptide linker” comprises in accordance with the present disclosure an amino acid sequence by which the amino acid sequences of one (variable and/or binding) domain and another (variable and/or binding) domain of the antibody construct of the disclosure are linked with each other. The peptide linkers can also be used to fuse the third domain to the other domains of the antibody construct of the disclosure. A feature of such peptide linker is that it does not comprise any polymerization activity. Among the suitable peptide linkers are those described in U.S. Pat. Nos. 4,751,180 and 4,935,233 or WO 88/09344, the disclosure of which are incorporated herein by reference in their entireties. The peptide linkers can also be used to attach other domains or modules or regions (such as half-life extending domains) to the bispecific antibody construct described herein.

In some embodiments, the third domain comprises a “Fc” or “Fc region” or “Fc domain,” which refers to the polypeptide comprising the constant region of an antibody excluding the first constant region immunoglobulin domain. Thus, “Fc domain” refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, the bispecific antibody construct is an IgG antibody (which includes several subclasses, including, but not limited to IgG1, IgG2, IgG3, and IgG4). Although the boundaries of the Fc region may vary, the human IgG heavy chain Fc region is usually defined to include residues C226 or P230 to its carboxyl-terminus, wherein the numbering is according to the EU index as in Kabat. In some embodiments, amino acid modifications are made to the Fc region, for example, to alter binding to one or more FcγR receptors or to the FcRn receptor.

In some embodiments, the formulations described herein comprise a bispecific antibody construct which binds human CD3 and human CDH19, or human CD3 and human MSLN, or human CD3 and human DLL3, or human CD3 and human FLT3, or human CD3 and human EGFRvIII, or human CD3 and human BCMA, or human CD3 and PSMA, or human CD3 and human CD33, or human CD3 and human CD19, human CD3 and human CD70, or human CD3 and human MUC17, or human CD3 and human CLDN18.2.

In some embodiments, the first binding domain of the bispecific antibody construct comprises a set of 6 CDRs set forth in (a) SEQ ID NOs: 24-29, (b) SEQ ID NOs: 34-39, (c) SEQ ID NOs: 78-83, (d) SEQ ID NOs: 10-15, (e) SEQ ID NOs: 46-51, (f) SEQ ID NOs: 88-93, (g) SEQ ID NOs: 67-72, (h) SEQ ID NOs: 56-61, (i) SEQ ID NOs: 112-117, (j) SEQ ID NOs: 100-105, (k) SEQ ID NOs:148-153, SEQ ID NOs: 157-162, or SEQ ID NOs: 166-171, or SEQ ID NOs: 175-180, (l) SEQ ID NOs:132-137, or (m) SEQ ID NOs: 123-128.

In some embodiments, the first binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 30, 40, 84, 16, 17, 52, 94, 73, 62, 118, 154, 163, 172, 181, 106, 138, 143, or 129. In some embodiments, the first binding domain of the bispecific antibody construct comprises a VH comprising the amino acid sequence set forth in SEQ ID NO: 30, 40, 84, 16, 17, 52, 94, 73, 62, 118, 154,163, 172, 181, 106, 138, 143, or 129.

In some embodiments, the first binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 31, 41, 85, 18, 19, 53, 95, 74, 63, 119, 155, 164, 173, 182, 107, 139, 144, or 130. In some embodiments, the first binding domain of the bispecific antibody construct comprises a VL comprising the amino acid sequence set forth in SEQ ID NO: 31, 41, 85, 18, 19, 53, 95, 74, 63, 119, 155, 164, 173, 182, 107, 139, 144, or 130.

In some embodiments, wherein the first binding domain comprises (a) a VH region comprising an amino acid sequence set forth in SEQ ID NO: 30 and a VL region comprising an amino acid sequence set forth in SEQ ID NO: 31; (b) a VH region comprising an amino acid sequence set forth in SEQ ID NO: 40 and a VL region comprising an amino acid sequence set forth in SEQ ID NO: 41; (c) a VH region comprising an amino acid sequence set forth in SEQ ID NO: 84 and a VL region comprising an amino acid sequence set forth in SEQ ID NO: 85; (d) a VH region comprising an amino acid sequence set forth in SEQ ID NO: 16 or 17 and a VL region comprising an amino acid sequence set forth in SEQ ID NO: 18 or 19; (e) a VH region comprising an amino acid sequence set forth in SEQ ID NO: 52 and a VL region comprising an amino acid sequence set forth in SEQ ID NO: 53; (f) a VH region comprising an amino acid sequence set forth in SEQ ID NO: 94 and a VL region comprising an amino acid sequence set forth in SEQ ID NO: 95; (g) a VH region comprising an amino acid sequence set forth in SEQ ID NO: 73 and a VL region comprising an amino acid sequence set forth in SEQ ID NO: 74; (h) a VH region comprising an amino acid sequence set forth in SEQ ID NO: 62 and a VL region comprising an amino acid sequence set forth in SEQ ID NO: 63; (i) a VH region comprising an amino acid sequence set forth in SEQ ID NO: 118 and a VL region comprising an amino acid sequence set forth in SEQ ID NO: 119; (j) a VH region comprising an amino acid sequence set forth in SEQ ID NO: 154, 163, 172, or 181 and a VL region comprising an amino acid sequence set forth in SEQ ID NO: 155, 164, 173 or 182; (k) a VH region comprising an amino acid sequence set forth in SEQ ID NO: 106 and a VL region comprising an amino acid sequence set forth in SEQ ID NO: 107; (l) a VH region comprising an amino acid sequence set forth in SEQ ID NO: 138 or 143, and a VL region comprising an amino acid sequence set forth in SEQ ID NO: 139 or 144; or (m) a VH region comprising an amino acid sequence set forth in SEQ ID NO: 129 and a VL region comprising an amino acid sequence set forth in SEQ ID NO: 130.

In some embodiments, the second binding domain of the bispecific antibody construct comprises a set of 6 CDRs set forth in SEQ ID NOs: 1-6.

In some embodiments, the second binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 7. In some embodiments, the second binding domain of the bispecific antibody construct comprises a VH comprising the amino acid sequence set forth in SEQ ID NO: 7.

In some embodiments, the second binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 8. In some embodiments, the second binding domain of the bispecific antibody construct comprises a VL comprising the amino acid sequence set forth in SEQ ID NO: 8.

In some embodiments, wherein the second binding domain comprises (a) a VH region comprising an amino acid sequence set forth in SEQ ID NO: 7 and a VL region comprising an amino acid sequence set forth in SEQ ID NO: 8.

In some embodiments, the bispecific antibody construct comprises a first binding domain that binds CD19 comprising an anti-CD19 variable light domain comprising the amino acid sequence of SEQ ID NO: 85 and an anti-CD19 variable heavy domain comprising the amino acid sequence of SEQ ID NO: 84, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 86 a second binding domain comprising the amino acid sequence of SEQ ID NO: 9. In some embodiments, the bispecific antibody construct comprises the amino acid sequence set forth in SEQ ID NO: 87.

In some embodiments, the bispecific antibody construct comprises a first binding domain that binds MSLN comprising an anti-MSLN variable light domain comprising the amino acid sequence of SEQ ID NO: 41 and an anti-MSLN variable heavy domain comprising the amino acid sequence of SEQ ID NO: 40, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 42, and a second binding domain comprising the amino acid sequence of SEQ ID NO: 9. In some embodiments, the bispecific antibody construct comprises an amino acid sequence set forth in SEQ ID NO: 43, 44 or 45.

In some embodiments, the bispecific antibody construct comprises a first binding domain that binds DLL3 comprising an anti-DLL3 variable light domain comprising the amino acid sequence of SEQ ID NO: 74 and an anti-DLL3 variable heavy domain comprising the amino acid sequence of SEQ ID NO: 73, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 75, and a second binding domain comprising the amino acid sequence of SEQ ID NO: 9. In some embodiments, the bispecific antibody construct comprises an amino acid sequence set forth in SEQ ID NO: 76 or 77.

In some embodiments, the bispecific antibody construct comprises a first binding domain that binds FLT3 comprising an anti-FLT3 variable light domain comprising the amino acid sequence of SEQ ID NO: 63 and an anti-FLT3 variable heavy domain comprising the amino acid sequence of SEQ ID NO: 62, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 64, a second binding domain comprising the amino acid sequence of SEQ ID NO: 9. In some embodiments, the bispecific antibody construct comprises an amino acid sequence set forth in SEQ ID NO: 65 or 66.

In some embodiments, the bispecific antibody construct comprises a first binding domain that binds EGFRvIII comprising an anti-EGFRvIII variable light domain comprising the amino acid sequence of SEQ ID NO: 31 and an anti-EGFRvIII variable heavy domain comprising the amino acid sequence of SEQ ID NO: 30, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 32, a second binding domain comprising the amino acid sequence of SEQ ID NO: 9. In some embodiments, the bispecific antibody construct comprises an amino acid sequence set forth in SEQ ID NO: 33.

In some embodiments, the bispecific antibody construct comprises a first binding domain that binds BCMA comprising an anti-BCMA variable light domain comprising the amino acid sequence of SEQ ID NO: 95 and an anti-BCMA variable heavy domain comprising the amino acid sequence of SEQ ID NO: 94, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 96, a second binding domain comprising the amino acid sequence of SEQ ID NO: 9. In some embodiments, the bispecific antibody construct comprises an amino acid sequence set forth in SEQ ID NO: 98 or SEQ ID NO: 97.

In some embodiments, the bispecific antibody construct comprises a first binding domain that binds PSMA comprising an anti-PSMA variable light domain comprising the amino acid sequence of SEQ ID NO: 119 or 107 and an anti-PSMA variable heavy domain comprising the amino acid sequence of SEQ ID NO: 118 or 106, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 120 or 108, a second binding domain comprising the amino acid sequence of SEQ ID NO: 9. In some embodiments, the bispecific antibody construct comprises an amino acid sequence set forth in SEQ ID NO: 121, 122, 109, 110 or 111.

In some embodiments, the bispecific antibody construct comprises a first binding domain that binds CD33 comprising an anti-CD33 variable light domain comprising the amino acid sequence of SEQ ID NO: 18 or 19 and an anti-CD33 variable heavy domain comprising the amino acid sequence of SEQ ID NO: 16 or 17, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 189 or 190, a second binding domain comprising the amino acid sequence of SEQ ID NO: 9. In some embodiments, the bispecific antibody construct comprises the amino acid sequence set forth in SEQ ID NO: 20, 21, 22 or 23.

In some embodiments, the bispecific antibody construct comprises a first binding domain that binds CDH19 comprising an anti-CDH19 variable light domain comprising the amino acid sequence of SEQ ID NO: 53 and an anti-CDH19 variable heavy domain comprising the amino acid sequence of SEQ ID NO: 52, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 54, a second binding domain comprising the amino acid sequence of SEQ ID NO: 9. In some embodiments, the bispecific antibody construct comprises the amino acid sequence set forth in SEQ ID NO: 55.

In some embodiments, the bispecific antibody construct comprises a first binding domain that binds MUC17 comprising an anti-MUC17 variable light domain comprising the amino acid sequence of SEQ ID NO: 155, 164, 173, or 182 and an anti-MUC17 variable heavy domain comprising the amino acid sequence of SEQ ID NO: 154, 163, 172, or 181 a second binding domain comprising an anti-CD3 variable heavy domain comprising the amino acid sequence of SEQ ID NO: 7, and an anti-CD3 variable light domain comprising the amino acid sequence of SEQ ID NO: 8. In some embodiments, the bispecific antibody construct comprises the amino acid sequence set forth in SEQ ID NO: 156, 165, 174 or 183.

In some embodiments, the bispecific antibody construct comprises a first binding domain that binds cldn18.2 comprising an anti-cldn18.2 variable light domain comprising the amino acid sequence of SEQ ID NO: 139 or 144 and an anti-cldn18.2 variable heavy domain comprising the amino acid sequence of SEQ ID NO: 138 or 143, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 140 or 145, and a second binding domain comprising the amino acid sequence of SEQ ID NO: 9. In some embodiments, the bispecific antibody construct comprises the amino acid sequence set forth in SEQ ID NO: 141, 142, 146 or 147.

In some embodiments, the bispecific antibody construct comprises a first binding domain that binds CD70 comprising an anti-CD70 variable light domain comprising the amino acid sequence of SEQ ID NO: 130 and an anti-CD70 variable heavy domain comprising the amino acid sequence of SEQ ID NO: 129, a second binding domain comprising an anti-CD3 variable heavy domain comprising the amino acid sequence of SEQ ID NO: 7, and an anti-CD3 variable light domain comprising the amino acid sequence of SEQ ID NO: 8. In some embodiments, the bispecific antibody construct comprises an amino acid sequence set forth in SEQ ID NO: 131.

In some embodiments, the protein of the formulation is an antibody. In various embodiments, the protein of the formulation is a bispecific antibody construct. In some cases, the protein of the formulation is a half-life extended bispecific antibody construct. Half-life extended bispecific antibody constructs have been previously described herein. In some embodiments, the protein formulation of the disclosure comprises an amino acid sequence set forth in SEQ ID NOs: 1-190. In various embodiments, the protein formulation of the disclosure comprises an amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 55, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188. In some cases, the protein formulation of the disclosure comprises an amino acid sequence set forth in SEQ ID NO: 22 (BiTE A), SEQ ID NO: 77 (BiTE B), SEQ ID NO: 87 (BiTE C), or SEQ ID NO: 97 (BiTE D).

In some embodiments, the protein, such an antibody or bispecific antibody construct (e.g., HLE bispecific antibody construct), is present in the liquid formulation (before lyophilization) in an amount ranging from about 0.1 mg/mL to about 100 mg/mL (or about 0.1 mg/mL, 0.5 mg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, or 100 mg/mL). In various embodiments, the protein is present in the liquid formulation in an amount ranging from about 0.1 mg/mL to about 70 mg/mL. In some cases, the protein is present in the liquid formulation in an amount ranging from about 0.5 mg/mL to about 30 mg/mL (or about 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, or 30 mg/mL). In various cases, the protein is present in the liquid formulation in an amount ranging from about 1 mg/mL to about 20 mg/mL (or about 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 11.5 mg/mL, 12 mg/mL, 12.5 mg/mL, 13 mg/mL, 13.5 mg/mL, 14 mg/mL, 14.5 mg/mL, 15 mg/mL, 15.5 mg/mL, 16 mg/mL, 16.5 mg/mL, 17 mg/mL, 17.5 mg/mL, 18 mg/mL, 18.5 mg/mL, 19 mg/mL, 19.5 mg/mL. or 20 mg/mL). In some embodiments, the protein is present in the liquid formulation in an amount of about 1 mg/mL.

Saccharide

The protein formulation of the disclosure comprises a saccharide. In some embodiments, the saccharide is a monosaccharide or a disaccharide. Suitable saccharides include, for example, glucose, galactose, fructose, xylose, sucrose, lactose, maltose, trehalose, or any combination thereof. In some cases, the saccharide comprises sucrose.

In some embodiments, the liquid formulation (before lyophilization) comprises saccharide at a concentration of about 1% to about 15% w/v, or about 4% to about 13% w/v, or about 6% to about 12% w/v. In some embodiments, the liquid formulation comprises saccharide at a concentration of at least 1%, at least 2%, at least 3%, at least 4%, at least 5%, at least 6%, at least 7%, at least 8%, at least 9%, at least 10%, at least 11%, at least 12%, at least 13%, or at least 14% w/v. In some embodiments, the liquid formulation comprises saccharide at a concentration of about 1%, about 2%, about 3%, about 4%, about 5%, about 6%, about 7%, about 8%, about 9%, about 10%, about 11%, about 12%, about 13%, about 14%, or about 15% w/v. In some embodiments, the liquid formulation comprises saccharide at a concentration of about 7%, about 7.5%, about 8%, about 8.5%, about 9%, about 9.5%, about 10%, about 10.5%, about 11%, about 11.5%, or about 12% w/v. In some embodiments, the liquid formulation comprises saccharide at a concentration of about 7% to about 12% w/v. In some embodiments, the liquid formulation comprises saccharide at a concentration of about 9% w/v. In some embodiments, the saccharide is sucrose and is present in the liquid formulation at a concentration ranging from about 6% to about 12% w/v. In some cases, the saccharide is sucrose and is present in the liquid formulation at a concentration of about 9% w/v.

Surfactant

The protein formulation of the disclosure comprises a surfactant. Suitable surfactants include a polysorbate, a poloxomer, a polyoxyethylene, or any combination thereof. Contemplated surfactants include polysorbate 20, polysorbate 40, polysorbate 60, polysorbate 80, poloxamer 188, poloxamer 407, triton X-100, polyoxyethylene, PEG 3350, PEG 4000, and any combination thereof. In some embodiments, the surfactant comprises a polysorbate. In some cases, the surfactant is polysorbate 80.

The protein formulations described herein can comprise one surfactant or a mixture of surfactants. In some embodiments, the liquid formulation (before lyophilization) comprises a surfactant at a concentration of about 0.001% to about 5% w/v (or about 0.001% to about 0.5%, or about 0.004 to about 0.5% w/v or about 0.001 to about 0.01% w/v or about 0.004 to about 0.01% w/v). In some embodiments, the liquid formulation comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some embodiments, the liquid formulation comprises a surfactant at a concentration of about 0.001% to about 0.5% w/v. In some embodiments, the liquid formulation comprises a surfactant at a concentration of about 0.001 to about 0.01% w/v. In some embodiments, the liquid formulation comprises a surfactant at a concentration of about 0.001 to about 0.01% w/v. In some embodiments, the liquid formulation comprises a surfactant at a concentration of about 0.001%, about 0.002%, about 0.003%, about 0.004%, about 0.005%, about 0.006%, about 0.007%, about 0.008%, about 0.009%, about 0.01%, about 0.05%, about 0.1%, about 0.2%, about 0.3%, about 0.4%, to about 0.5% w/v. In some embodiments, the liquid formulation comprises a surfactant at a concentration of about 0.001% to about 0.01% w/v. In some embodiments, the surfactant is polysorbate 80 and the polysorbate 80 is present in a concentration of about 0.01% w/v.

Buffer

The protein formulation of the disclosure optionally comprises a buffer. Suitable buffers include acetate buffers, glutamate buffers, citrate buffers, lactate buffers, succinate buffers, tartrate buffers, fumarate buffers, maleate buffers, histidine buffers, phosphate buffers, 2-(N-morpholino)ethanesulfonate buffers, or any combination thereof. In some cases, the buffer comprises glutamic acid.

Buffering agents are often employed to control pH in the formulation. In some embodiments, the buffer is added in a concentration that maintains pH of the liquid formulation of about 3 to about 7, or about 4 to about 6, about 4 to 5, or about 4.2. The effect of pH on formulations may be characterized using any one or more of several approaches such as accelerated stability studies and calorimetric screening studies (Remmele R. L. Jr., et al., Biochemistry, 38(16): 5241-7 (1999)).

The buffer system present in the protein formulation is selected to be physiologically compatible and to maintain a desired pH. The buffer may be present in the liquid formulation (before lyophilization) at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some embodiments, the buffer in the liquid protein formulation (before lyophilization) is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM. In some embodiments, the concentration of the buffer is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some embodiments, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some embodiments, the concentration of the buffer is between 5, 6, 7, 8, 9, 10, 11, 12, 13, 14, 15, 16, 17, 18, 19, 20, 30, or 40 mM and 50 mM. In some embodiments, the concentration of the buffer is about 10 mM.

In some embodiments, the liquid protein formulation (before lyophilization) has a pH of about 4.2 and comprises about 10 mM L-glutamic acid, about 9.0% (w/v) sucrose, and about 0.01% (w/v) polysorbate 80.

### Stability of Lyophilized Protein Formulation

The methods disclosed herein advantageously result in a lyophilized protein formulation that exhibits decreased physical degradation, such as aggregation, as well as decreased chemical degradation, such as decreased clipping and deamidation, of the protein upon reconstitution with a liquid. The liquid used for reconstituting the lyophilized protein formulation can be any suitable liquid known in the art. In embodiments, the lyophilized protein formulation can be reconstituted with water. Furthermore, the lyophilization methods disclosed herein are able to stabilize both low and high concentration protein formulations, such as formulations containing antibodies and bispecific antibody constructs (e.g., half-life extended bispecific antibody constructs).

The stability of a protein formulation, such as a formulation containing an antibody or a bispecific antibody construct (e.g., a HLE bispecific antibody construct), can be quantified in several ways. In some embodiments, stability of a protein formulation is characterized by size exclusion high performance liquid chromatography (SE-HPLC), size exclusion ultra-high performance liquid chromatography (SE-UHPLC), cation exchange high performance liquid chromatography (CE-HPLC), dynamic light scattering (DLS), analytical ultracentrifugation (AUC), field flow fractionation (FFF), isoelectric focusing and ion exchange chromatography (IEX). In some embodiments, stability of protein formulation, such as an antibody formulation, is characterized by partial dissociation as measured by sodium-dodecyl sulfate capillary electrophoresis (CE-SDS) and/or sodium-dodecyl sulfate polyacrylamide gel electrophoresis (SDS-PAGE). In some embodiments, stability of the formulation is assessed by reduced capillary electrophoresis-sodium dodecyl sulfate (rCE-SDS). The rCE-SDS method separates the heavy chain (HC), light chain (LC), non-glycosylated HC (NGHC), and other minor peak species and groups under reducing conditions.

In some embodiments, stability of the formulation is characterized by the amount of high molecular weight (HMW) species of a protein, such as an antibody or bispecific antibody construct (e.g., HLE bispecific antibody construct), or by the rate of increase of the amount of HMW species of the protein after storage conditions at various time points. In some embodiments, the amount of HMW species of the protein is determined after one week, two weeks, one months, three months, six months or twelve months in storage at approximately 4° C. or 40° C. after reconstitution. In some embodiments, the rate of increase of HMW species of the protein is determined after one week, two weeks, one month, three months, six months or twelve months in storage at approximately 4° C. or 40° C. after reconstitution. In some embodiments, the HMW species of a protein, such as an antibody or bispecific antibody construct (e.g., HLE bispecific antibody construct), in the reconstituted lyophilized formulation is measured by SE-UHPLC.

The stability of a protein, such as an antibody or bispecific antibody construct (e.g., HLE bispecific antibody construct), and the capability of the formulation to maintain stability of the protein, may be assessed over extended periods of time (e.g., weeks or months). In the context of a formulation, a stable formulation is one in which the protein, such as an antibody or bispecific antibody construct (e.g., HLE bispecific antibody construct), therein essentially retains its physical and/or chemical integrity and/or biological activity upon storage and during processes such as freeze/thaw, mechanical mixing and lyophilization. Protein stability can be assessed, for example, by measuring the level and/or rate of formation of high molecular weight (HMW) aggregates, shift of charge profiles, and change in particle size.

In some embodiments, the relative values of any particular species of a protein, such as the intact BiTE® molecule or main species, or the high molecular weight (HMW) species (i.e., aggregates), or the low molecular weight (LMW) species (i.e., fragments), are expressed in relation to the respective values of the total product. For example, in some embodiments, 2.5% or less (e.g., 2.5%, or 2%, or 1.9%, or 1.8%, or 1.7%, or 1.6%, or 1.5%, or 1.4%, or 1.3%, or 1.2%, or 1.1%, or 1%, or 0.5%) of the protein, such as the antibody or bispecific antibody construct, exists as HMW species in the reconstituted lyophilized formulation. In some embodiments, the amount of HMW species in the reconstituted lyophilized formulation increases less than 1%, (e.g., 0.9%, 0.8%, 0.7%, 0.6%, 0.5%, 0.4%, 0.3%, 0.2%, 0.1%) upon storage at 4° C. for one month or more (e.g., for one month, for three months, or for six months). In some embodiments, upon storage at 4° C. for one month or more (e.g., for one month, for three months, or for six months), the amount of HMW species in the reconstituted lyophilized formulation increases approximately between 0.1% and 0.4% (e.g., 0.1%, 0.2%, 0.3%, or 0.4%). In some embodiments, the amount of HMW species in the reconstituted lyophilized formulation increases less than 1%, (e.g., 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, the amount of HMW species in the reconstituted lyophilized formulation increases less than 0.5%, (e.g., 0.5%, 0.4%, 0.3%, 0.2%, 0.1%) upon storage at 40° C. for one week or more (e.g., for one week, for two weeks, for one month or for three months). In some embodiments, the amount of HMW species in the reconstituted lyophilized formulation increases less than 0.5%, (e.g., 0.5%, 0.4%, 0.3%, 0.2%, 0.1%) upon storage at 40° C. for one month or more (e.g., for one month, for three months, for six months, for nine months, or for twelve months). In some embodiments, the amount of HMW species in the reconstituted lyophilized formulation increases less than 0.5% upon storage at 40° C. for one month. In some embodiments, the amount of HMW species in the reconstituted lyophilized formulation increases less than 0.3% upon storage at 40° C. for one month. In some embodiments, upon storage at 40° C. for one week or more (e.g., for one week, for two weeks, for one month or for three months) the amount of HMW species in the reconstituted lyophilized formulation increases approximately between 0.1% and 0.7% (e.g., 0.1%, 0.2%, 0.3%, 0.4%, 0.5%, 0.6%. or 0.7%). In some embodiments, upon storage at 40° C. for one week or more (e.g., for one week, for two weeks, for one month or for three months) the amount of HMW species in the reconstituted lyophilized formulation increases approximately between 0.1% and 0.5% (e.g., 0.1%, 0.2%, 0.3%, 0.4%, and 0.5%). In some embodiments, upon storage at 40° C. for one month or more (e.g., for one month, for three months, for six months, for nine months, or for twelve months) the amount of HMW species in the reconstituted lyophilized formulation increases approximately between 0.1% and 0.5% (e.g., 0.1%, 0.2%, 0.3%, 0.4%, and 0.5%). In some embodiments, the HMW species of a bispecific antibody construct in the reconstituted lyophilized formulation is measured by SE-UHPLC.

In some embodiments, stability of the formulation is characterized by the amount of low molecular (LMW) species of a protein, such as an antibody or bispecific antibody construct (HLE bispecific antibody construct), or by the rate of increase of the amount of LMW species of the protein under storage conditions at various time points. In some embodiments, the amount of LMW species is determined at one week, two weeks, one months, three months, six months or twelve months in storage at approximately 4° C. or 40° C. In some embodiments, the rate of increase of LMW species is determined at one week, two weeks, one month, three months, six months or twelve months in storage at approximately 4° C. or 40° C. In some embodiments, the LMW species of a protein, such as an antibody or bispecific antibody construct (HLE bispecific antibody construct), in the formulation is measured by reduced capillary electrophoresis-sodium dodecyl sulfate (rCE-SDS). In some embodiments, the LMW species of a bispecific antibody construct in the formulation is measured by Size Exclusion Chromatography (SEC).

In some embodiments, less than 2%, (e.g., 1.9%, 1.8%, 1.7%, 1.6%, 1.5%, 1.4%, 1.3%, 1.2%, 1.1%, 1%, or 0.5%) of the protein, such as an antibody or bispecific antibody construct (HLE bispecific antibody construct), exists as low molecular weight (LMW) species in the reconstituted lyophilized formulation. In some embodiments, the amount of LMW species in the reconstituted lyophilized formulation increases less than 2%, (e.g., 1.9%, 1.8%, 1.7%, 1.6%, 1.5%, 1.4%, 1.3%, 1.2%, 1.1%, 1%, or 0.5%) upon storage at 4° C. for one month or more (e.g., for one month, for three months, or for six months). In some embodiments, upon storage at 4° C. for one month or more (e.g., for one month, for three months, or for six months), the amount of LMW species in the reconstituted lyophilized formulation increases approximately between 0.1% and 0.7% (e.g., 0.1%, 0.2%, 0.3%, 0.4%, 0.5%, 0.6%. or 0.7%). In some embodiments, the amount of LMW species in the reconstituted lyophilized formulation increases less than 1%, (e.g., 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of LMW species in the reconstituted lyophilized formulation increases approximately between 0.1% and 0.7% (e.g., 0.1%, 0.2%, 0.3%, 0.4%, 0.5%, 0.6%. or 0.7%). In some embodiments, the LMW species of a bispecific antibody construct in the reconstituted lyophilized formulation is measured by Size Exclusion Chromatography (SEC). In some embodiments, the LMW species of a bispecific antibody construct in the reconstituted lyophilized formulation is measured by reduced capillary electrophoresis-sodium dodecyl sulfate (rCE-SDS).

In some embodiments, the percent of protein, such as an antibody or bispecific antibody construct (HLE bispecific antibody construct) (i.e., main peak species) in the reconstituted lyophilized formulation is greater than 95% of the total protein content in the formulation.

In some embodiments, the formulation is stable upon storage at about 4° C. for one month, and the amount of HMW species in the reconstituted lyophilized formulation increases approximately between 0.1% to 0.7% (e.g., 0.1%, or 0.2%, or 0.3%, or 0.4%, or 0.5%, or 0.6%, or 0.7%), while in storage for at least one month. In some embodiments, the formulation is stable upon storage at about 4° C. for three months, and the amount of HMW species in the reconstituted lyophilized formulation increases approximately between 0.0% to 0.2% (e.g., 0%, or 0.1%, or 0.2%), while in storage for at least three months. In some embodiments, the formulation is stable upon storage at about 4° C. for six months, and the amount of HMW species in the reconstituted lyophilized formulation increases approximately between 0.0% to 0.4% (e.g., 0%, or 0.1%, or 0.2%, or 0.3%, or 0.4%), while in storage for at least six months. In some embodiments, the HMW species of a bispecific antibody construct in the reconstituted lyophilized formulation is measured by SE-UHPLC.

In some embodiments, the formulation is stable upon storage at about 4° C. for one month, three months and six months, and the percent of protein, such as an antibody or bispecific antibody construct (HLE bispecific antibody construct), is above 95% of the total protein content. In some embodiments, the formulation is stable upon storage at about 4° C. for one month, three months, six months, twelve months, and 48 months, and the percent of protein, such as an antibody or bispecific antibody construct (HLE bispecific antibody construct), is above 96% of the total protein content after reconstitution.

The stability of a formulation described herein can also be characterized by charge distribution, e.g., a change in the amount of the charge variant peaks of the protein, such as an antibody or bispecific antibody construct (HLE bispecific antibody construct). For example, in some embodiments, the amount of acidic peak (e.g., deamidation, charge variants having a relatively lower isolectric point (pl)) in the reconstituted lyophilized formulation increases by less than 2% (e.g., 2%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, or less) when stored at 4° C. for at least one month (e.g., for one month, three months, six months or twelve months). In some embodiments, the amount of basic peak (e.g., charge variants having a relatively higher pl) in the reconstituted lyophilized formulation increases by less than 6% (e.g., 6%, 5%, 4%, 3%, 2% or 1%) when stored at 4° C. for at least one month (e.g., for one month, three months, six months or twelve months). In some embodiments, the amount of main peak in the reconstituted lyophilized formulation decreases by less than 4% (e.g., 4%, 3.5%, 3%, 2.5%, 2% 1% or less) when stored at 4° C. for at least one month. In some embodiments, the amount of main peak in the reconstituted lyophilized formulation decreases by less than 6% (e.g., 6%, 5%, 4%, 3.5%, 3%, 2.5%, 2% or less) when stored at 4° C. for at least three months. In some embodiments, the amount of main peak in the reconstituted lyophilized formulation decreases by less than 9% (e.g., 9%, 8%, 7%, 6%, 5%, 4%, 3.5%, 3%, 2.5%, 2% or less) when stored at 4° C. for at least six months. In some embodiments, the amount of main peak in the reconstituted lyophilized formulation decreases by less than 9% (e.g., 9%, 8%, 7%, 6%, 5%, 4%, 3.5%, 3%, 2.5%, 2% or less) when stored at 4° C. for at least twelve months.

In some embodiments, the amount of acidic peak in the reconstituted lyophilized formulation increases by less than 30% (e.g., 30%, 25%, 20%, 15%, 10%, 9%, 8%, 7%, 6%, 4%, 4%, 3%, 2%, 1% or less) when stored at 40° C. for at least one week (e.g., for one week, two weeks, one month or three months). In some embodiments, the amount of basic peak (e.g., charge variants having a relatively higher pl) in the reconstituted lyophilized formulation increases by less than 15% (e.g., 15%, 10%, 9%, 8%, 7%, 6%, 4%, 4%, 3%, 2%, 1% or less), when stored at 40° C. for at least one week (e.g., for one week, two weeks, one month or three months). In some embodiments, the amount of main peak in the reconstituted formulation decreases by less than 4% (e.g., 4%, 3.5%, 3%, 2.5%, 2% 1% or less), when stored at 4° C. for at least one month. In some embodiments, the amount of main peak in the reconstituted lyophilized formulation decreases by less than 6% (e.g., 6%, 5%, 4%, 3.5%, 3%, 2.5%, 2% or less), when stored at 4° C. for at least three months.

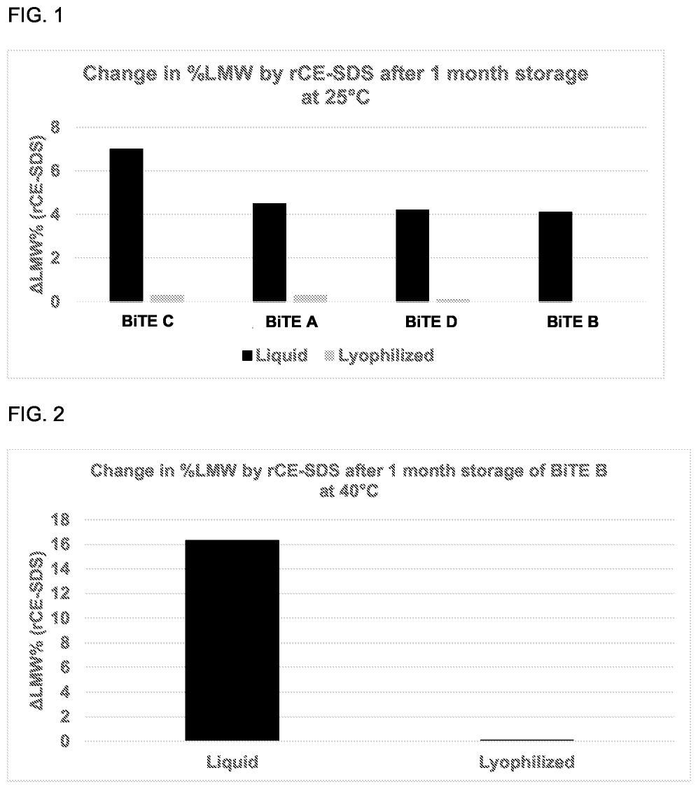

Protein formulations lyophilized by the methods of the disclosure exhibit superior stability over comparable liquid protein formulations. For example, the stability of protein formulations containing 1 mg/mL of a bispecific antibody construct of the disclosure, 10 mM L-glutamic acid, 9% (w/v) sucrose, and 0.01% (w/v) polysorbate 80, at pH 4.2 that were lyophilized according to the disclosure using an annealing step and then reconstituted were subjected to reduced capillary electrophoresis with sodium dodecyl sulfate (rCE-SDS) to determine the degree of clipping that occurred after one month of storage at 25° C. and at 40° C. See Example 3. As shown in FIGS. 1 and 2, the reconstituted lyophilized formulations exhibited significantly less clipping than the liquid formulations at both temperatures, demonstrating that protein formulations lyophilized according to the methods described herein have superior stability over comparable liquid formulations.

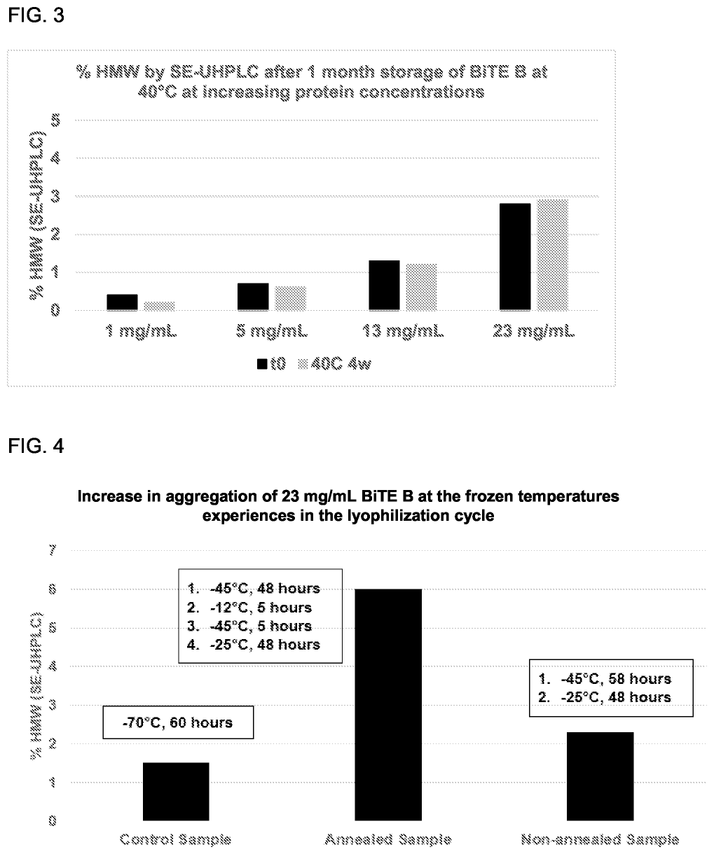

The lyophilization methods of the disclosure also advantageously stabilize protein formulations at both low and high concentrations. For example, protein formulations of the disclosure containing 1 mg/mL, 5 mg/mL, 13 mg/mL, and 23 mg/mL of a bispecific antibody construct of the disclosure, 10 mM L-glutamic acid, 9% (w/v) sucrose, and 0.01% (w/v) polysorbate 80, at pH 4.2 that had been lyophilized using an annealing step and then reconstituted were subjected to SEC-UHPLC after one month of storage at 40° C. to determine the degree of aggregation in the formulation by the percentage of high molecular weight species (% HMW). See Example 4. As shown in FIG. 3, no increase in % HMW under accelerated stress conditions occurred, demonstrating that the lyophilization methods disclosed herein are able to stabilize formulations having both low and high concentrations of proteins, such as bispecific antibody constructs.

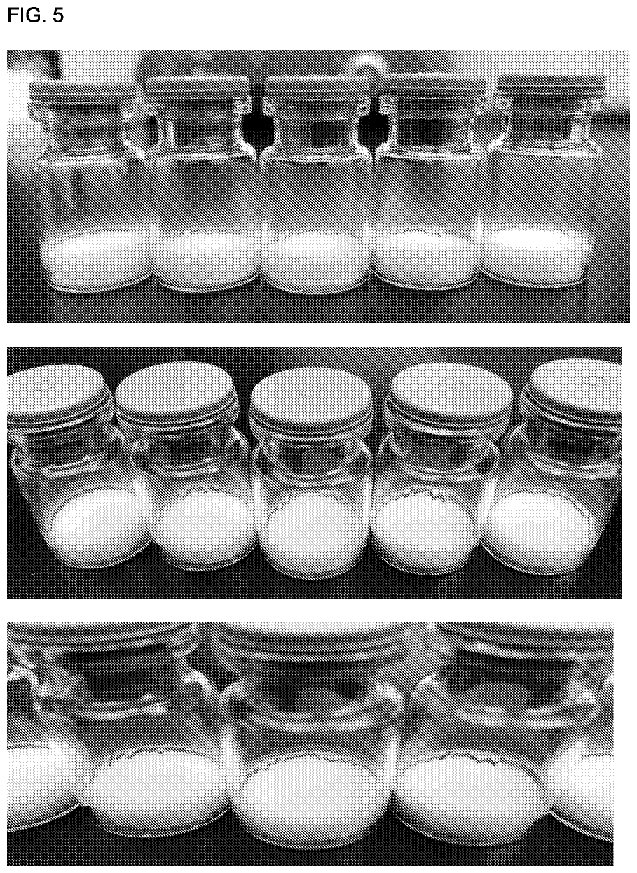

The lyophilization methods of the disclosure that lacked an annealing step were surprisingly found to result in superior stability of the protein formulation over lyophilization methods that included an annealing step. For example, protein formulations containing 15 mg/mL, 20 mg/mL, or 23 mg/mL of a bispecific antibody construct of the disclosure, 10 mM L-glutamic acid, 9% (w/v) sucrose, and 0.01% (w/v) polysorbate 80, at pH 4.2 that were subjected to lyophilization with and without an annealing step were subjected to SE-UHPLC after reconstitution to determine the amount of aggregation in each sample. See Example 5. As shown in Table 1, Table 2, FIG. 4, and FIG. 5, the sample that had been annealed exhibited significantly more aggregation, evidenced by a higher % HMW, than a sample that had not been annealed or a control sample.

The following examples are provided for illustration and are not intended to limit the scope of the invention.

### EXAMPLES

General Procedures

Reduced Capillary Electrophoresis with Sodium Dodecyl Sulfate (rCE-SDS) separates proteins based on differences in their hydrodynamic size under reducing and denaturing conditions. The protein species are bound to SDS, an anionic detergent, and electrokinetically injected into a bare fused silica capillary filled with SDS gel buffer. An electric voltage is applied across the capillary, under which the SDS coated proteins are separated by their difference in migration in a hydrophilic polymer-based solution. Proteins are detected by a photodiode array (PDA) detector as they pass through a UV detection window. Purity is evaluated by determining the percent corrected peak area of each component. The rCE-SDS method separates the heavy chain (HC), light chain (LC), non-glycosylated HC (NGHC), and other minor peak species and groups under reducing conditions. Reduced capillary electrophoresis with sodium dodecyl sulfate (rCE-SDS) was performed by incubating samples in an SDS-MW reducing gel for 10 minutes at 79° C. Following incubation, samples were centrifuged and then electrokinetically injected onto a 67-cm bare fused silica capillary having a 50 μm inner diameter using electrokinetic injection. The effective length of the capillary was 30.2 cm. Separation was performed using CE-SDS gel (Beckman Coulter, Brea, Calif.) and 30 kV effective voltage. Detection was performed at 220 nm by UV absorbance.

Size Exclusion Ultra High Performance Liquid Chromatography (SE-UHPLC). SEC-UHPLC separates proteins based on differences in their hydrodynamic volumes. Molecules with higher hydrodynamic volumes elute earlier than molecules with smaller volumes. The samples are loaded onto an SE-UHPLC column (BEH200, 4.6×300 mm, (Waters Corporation, 186005226)), separated isocratically and the eluent is monitored by UV absorbance. Purity is determined by calculating the percentage of each separated component as compared to the total integrated area. SE-UHPLC settings are as follows: Flow rate: 0.4 mL/min, Run time: 12 min, UV detection: 280 nm, Column temperature: Ambient, Target protein load: 6 μg, Protein compatible flow cell: 5 mm.

Protein Formulation. Protein formulations were prepared comprising an intact bispecific antibody construct at a concentration of 1 mg/mL, 5 mg/mL, 13 mg/mL, or 23 mg/mL, 10 mM L-glutamic acid, 9% (w/v) sucrose, 0.01% (w/v) polysorbate 80, at pH 4.2. The protein formulation was introduced into a vial for lyophilization (either with or without an annealing step).

### Example 1

### Lyophilization of Bispecific Antibody Construct Formulation with an Annealing Step

A liquid protein formulation was prepared as described above and introduced into a lyophilization chamber. The chamber was cooled from ambient temperature to −45° C. at a rate of 0.5° C./min, and held at −45° C. for 2 hours. The pressure of the lyophilization chamber was lowered from ambient to 70 mTorr, the chamber was heated to −25° C. at a rate of 0.33° C./min, and held at −25° C. for 52 hours at 70 mTorr pressure. The chamber was then heated to 30° C. at a rate of 0.1° C./min, and held at 30° C. for 8 hours at 70 mTorr. To enable vial stoppering, the temperature of the chamber was lowered to 5° C., and the chamber was aerated with nitrogen at 500 mTorr. The vial containing the lyophilized protein formulation was removed from the lyophilization chamber and stored at 2-8° C. until further processing and analysis.

The lyophilized protein formulation was reconstituted with water for injection.

### Example 2

### Lyophilization of Bispecific Antibody Construct Formulation without an Annealing Step

A liquid protein formulation was prepared as described above and introduced into a lyophilization chamber. The chamber was cooled from ambient temperature to −45° C. at a rate of 0.5° C./min, and held at −45° C. for 2 hours. The temperature of the chamber was ramped to −12° C. at a rate of 0.5° C./min, and held at −12° C. for 5 hours. The chamber was then cooled back to −45° C. at a rate of 0.5° C./min, and held at −45° C. for 2 hours. The pressure of the chamber was lowered from ambient to 70 mTorr, the chamber was heated to −25° C. at a rate of 0.33° C./min, and held at −25° C. for 52 hours at 70 mTorr pressure. The chamber was then heated to 30° C. at a rate of 0.1° C./min, and held at 30° C. for 8 hours at 70 mTorr. To enable vial stoppering, the temperature of the chamber was lowered to 5° C., and the lyophilization chamber was aerated with nitrogen at 500 mTorr. The vial containing the lyophilized protein formulation was removed from the lyophilization chamber and stored at 2-8° C. until further processing and analysis.

The lyophilized protein formulation was reconstituted with water for injection.

### Example 3

### Comparison of the Stability of Liquid and Lyophilized Formulations of Bispecific Antibody Constructs Using rCE-SDS

Liquid formulations and lyophilized formulations (prepared with an annealing step) containing 1 mg/ml of BiTE A, BiTE B, BiTE C, or BiTE D were prepared as described above. The liquid and reconstituted lyophilized (reconstituted with water for injection) formulations at a protein concentration of 1 mg/mL were subjected to reduced capillary electrophoresis with sodium dodecyl sulfate (rCE-SDS) to determine the degree of clipping that occurred after one month of storage at 25° C. As shown in FIG. 1, all of the reconstituted lyophilized formulations demonstrated significantly less clipping than the liquid formulations, as evidenced by a lower percentage of low molecular weight species (LMS %), which indicates that the reconstituted lyophilized formulations have superior stability over liquid formulations. Further, liquid and reconstituted lyophilized formulations containing 1 mg/ml of BiTE B were subjected to rCE-SDS to determine the degree of clipping that occurred after one month of storage at 40° C. As shown in FIG. 2, the reconstituted lyophilized formulation demonstrated significantly less clipping than the liquid formulation, as evidenced by a lower LMS %, which indicates that the lyophilized formulation (prepared using an annealing step) has superior stability over the liquid formulation even at a higher temperature.

### Example 4

### Effect of Concentration of the Stability of Lyophilized Formulations Using SE-UHPLC

Lyophilized formulations (prepared with an annealing step) having different concentrations of BiTE B (1 mg/mL, 5 mg/mL, 13 mg/mL, and 23 mg/mL) were subjected to SEC-UHPLC upon reconstitution after one month of storage at 40° C. to determine the degree of aggregation in the formulation, which was determined by the percentage of high molecular weight species (% HMW). As shown in FIG. 3, there was no increase in % HMW under accelerated stress conditions, demonstrating that the lyophilization cycle is able to stabilize formulations having both low and high concentrations of bispecific antibody constructs.

### Example 5

### Comparison of the Stability of Annealed and Non Annealed Lyophilized Formulations of a Bispecific Antibody Construct Using SE-UHPLC

The amount of aggregation in annealed (prepared according to Example 1) and non-annealed (prepared according to Example 2) samples of lyophilized formulations containing 23 mg/ml of BiTE B after storage at frozen temperatures was determined using SE-UHPLC. The annealed sample was stored as follows: 45° C. for 48 hours, −12° C. storage for 5 hours, −45° C. for 5 hours, and −25° C. for 48 hours. The non-annealed sample was stored at −46° C. for 58 hours followed by −25° C. for 48 hours. As shown in FIG. 4, the annealed sampled exhibited significantly more aggregation upon reconstitution, evidenced by a higher % HMW, than a non-annealed sample upon reconstitution or a control sample containing 10 mM glutamic acid, 9% (w/v/sucrose, 0.01% (w/v) polysorbate 80 with a drying temperature of −10° C. See FIG. 5.

The amount of aggregation in annealed (prepared according to Example 1) and non-annealed (prepared according to Example 2) samples of lyophilized formulations containing 15 mg/ml of BiTE B was determined using SE-UHPLC before and after the lyophilization occurred. As shown in Table 1, below, the formulation that had been annealed during the lyophilization process exhibited significantly more aggregation upon reconstitution, as evidenced by a greater % HMW, than the sample that had not been annealed during the lyophilization process upon reconstitution.

TABLE 1
% HMW (15 mg/ml of BiTE B)

| | Pre- | Post-lyophilization |
|---|---|---|
| | lyophilization | (after reconstitution) |
| Lyophilization cycle with | 0.437 | 1.182 |
| annealing (n = 1) | | |
| Lyophilization cycle; without | 0.820 | 0.788 |
| annealing (n = 1) | | |

The amount of aggregation in annealed (prepared according to Example 1) and non-annealed (prepared according to Example 2) samples of lyophilized formulations containing 20 mg/ml of BiTE A, BiTE C, or BiTE E (a bispecific antibody construct having a sequence set forth is SEQ ID NO: 122) was determined using SE-UHPLC before and after the lyophilization occurred. As shown in Table 2, below, reconstituted formulations that had been annealed during the lyophilization process exhibited significantly more aggregation, as evidenced by a greater % HMW, than reconstituted samples that had not been annealed during the lyophilization process.

TABLE 2

| Bispecific | | | | |
|---|---|---|---|---|
| Antibody | | | | |
| Construct | | | | |
| Having a | | | | |
| Sequence Set | | | | |
| Forth In | Condition | % HMW | % Main | % LMW |
| BiTE E | Pre-Lyophilization | 2.37 | 96.19 | 1.44 |
| | Post-Lyophilization | 3.35 | 95.23 | 1.42 |
| | (with annealing) | | | |
| | after reconstitution | | | |
| | Post-Lyophilization | 2.38 | 96.24 | 1.37 |
| | (no annealing) after | | | |
| | reconstitution | | | |
| BiTE C | Pre-Lyophilization | 1.61 | 96.23 | 2.16 |
| | Post-Lyophilization | 4.78 | 92.95 | 2.27 |
| | (with annealing) after | | | |
| | reconstitution | | | |
| | Post-Lyophilization | 1.89 | 96.45 | 1.66 |
| | (no annealing) after | | | |
| | reconstitution | | | |
| BiTE A | Pre-Lyophilization | 3.28 | 95.59 | 1.13 |
| | Post-Lyophilization | 4.29 | 94.55 | 1.15 |
| | (with annealing) after | | | |
| | reconstitution | | | |
| | Post-Lyophilization | 3.39 | 95.44 | 1.17 |
| | (no annealing) after | | | |
| | reconstitution | | | |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

It should be understood that when describing a range of values, the characteristic being described could be an individual value found within the range. For example, “a pH from about pH 4 to about pH 6,” could be, but is not limited to, pH 4, 4.2, 4.6, 5.1, 5.5 etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH.

When the term “about” is used, it means the recited number plus or minus 5%, 10%, 15% or more of that recited number. The actual variation intended is determinable from the context.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A method of preparing a lyophilized formulation, the method comprising:
(a) cooling a lyophilization chamber containing a liquid formulation comprising a protein, a saccharide, and a surfactant to a temperature ranging from about −35° C. to about −50° C. to produce a frozen formulation, and holding the chamber at a temperature ranging from about −40° C. to about −50° C. for a time period of about 2 hours to about 24 hours;
(b) heating the chamber to a temperature ranging from about −30° C. to about −20° C. and a pressure ranging from about 25 mTorr to about 100 mTorr to produce a primary dried formulation, and holding the chamber at a temperature ranging from about −30° C. to about −20° C. and a pressure ranging from about 25 mTorr to about 100 mTorr for a time period of about 45 hours to about 60 hours;
(c) heating the chamber to a temperature ranging from about 20° C. to about 35° C. to produce a secondary dried formulation, and holding the chamber at a temperature ranging from about 20° C. to about 30° C. and a pressure ranging from about 25 mTorr to about 100 mTorr for a time period of about 5 hours to about 10 hours to produce the lyophilized formulation;
wherein the liquid formulation has a pH of about 3-7 and does not contain mannitol; and the method lacks an annealing step.

2. (canceled)

3. The method of claim 1, wherein the cooling of step (a) occurs at a rate ranging from about 0.5° C./min to about 1° C./min.

4. (canceled)

5. (canceled)

6. (canceled)

7. (canceled)

8. (canceled)

9. The method of claim 1, wherein the heating of step (b) occurs at a rate ranging from about 0.1° C./min to about 1° C./min

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The method of claim 1, wherein the heating of step (c) occurs at a rate ranging from about 0.05° C./min to about 0.5° C./min.

22. (canceled)

23. (canceled)

24. (canceled)

25. (canceled)

26. (canceled)

27. The method of claim 1, wherein the protein is an antibody.

28. The method of claim 1, wherein the protein is a bispecific antibody construct.

29. The method of claim 28, wherein the bispecific antibody construct is a half-life extended (HLE) bispecific antibody construct.

30. The method of claim 29, wherein the HLE bispecific antibody construct comprises an amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 55, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

31. (canceled)

32. The method of claim 1, wherein the protein is present in the liquid formulation at a concentration ranging from about 0.1 mg/mL to about 100 mg/mL.

33. (canceled)

34. (canceled)

35. (canceled)

36. (canceled)

37. The method of claim 1, wherein the liquid formulation of step (a) has a pH of about 4-6.

38. The method of claim 1, wherein the liquid formulation of step (a) further comprises a buffer.

39. (canceled)

40. (canceled)

41. The method of claim 1, wherein the buffer is present at a concentration ranging from about 5 mM to about 200 mM.

42. (canceled)

43. (canceled)

44. The method of claim 1, wherein the saccharide is a monosaccharide or a disaccharide.

45. The method of claim 44, wherein the saccharide is glucose, galactose, fructose, xylose, sucrose, lactose, maltose, trehalose, or any combination thereof.

46. (canceled)

47. The method of claim 1, wherein the saccharide is present in the liquid formulation at a concentration ranging from about 1 to about 15% (w/v).

48. (canceled)

49. (canceled)

50. The method of claim 1, wherein the surfactant is polysorbate 20, polysorbate 40, polysorbate 60, polysorbate 80, poloxamer 188, poloxamer 407, triton X-100, polyoxyethylene, PEG 3350, PEG 4000, or a combination thereof.

51. (canceled)

52. The method of claim 1, wherein the surfactant is present in the liquid formulation at a concentration ranging from about 0.001% to 0.5% (w/v).

53. (canceled)

54. (canceled)

55. (canceled)

56. The method of claim 1, wherein the liquid formulation of step (a) has a pH of about 4.2 and comprises about 10 mM L-glutamic acid, about 9.0% (w/v) sucrose, and about 0.010% (w/v) polysorbate 80.

57. The method of claim 1, wherein the lyophilized formulation, upon reconstitution, exhibits a 0.5% or less increase in the percentage of high molecular weight species after storage for one month at 40° C.

58. (canceled)

59. A lyophilized protein formulation prepared by the method according to claim 1.


SEQUENCE LISTING (190 sequences)

SEQ ID NO: 1 - protein, 14 aa, Artificial Sequence
  Synthetic Polypeptide
GSSTGAVTSGNYPN

SEQ ID NO: 2 - protein, 7 aa, Artificial Sequence
  Synthetic Polypeptide
GTKFLAP

SEQ ID NO: 3 - protein, 9 aa, Artificial Sequence
  Synthetic Polypeptide
VLWYSNRWV

SEQ ID NO: 4 - protein, 5 aa, Artificial Sequence
  Synthetic Polypeptide
KYAMN

SEQ ID NO: 5 - protein, 19 aa, Artificial Sequence
  Synthetic Polypeptide
RIRSKYNNYATYYADSVKD

SEQ ID NO: 6 - protein, 14 aa, Artificial Sequence
  Synthetic Polypeptide
HGNFGNSYISYWAY

SEQ ID NO: 7 - protein, 125 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 8 - protein, 109 aa, Artificial Sequence
  Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 9 - protein, 249 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 10 - protein, 17 aa, Artificial Sequence
  Synthetic Polypeptide
KSSQSVLDSSTNKNSLA

SEQ ID NO: 11 - protein, 7 aa, Artificial Sequence
  Synthetic Polypeptide
WASTRES

SEQ ID NO: 12 - protein, 9 aa, Artificial Sequence
  Synthetic Polypeptide
QQSAHFPIT

SEQ ID NO: 13 - protein, 5 aa, Artificial Sequence
  Synthetic Polypeptide
NYGMN

SEQ ID NO: 14 - protein, 17 aa, Artificial Sequence
  Synthetic Polypeptide
WINTYTGEPTYADKFQG

SEQ ID NO: 15 - protein, 13 aa, Artificial Sequence
  Synthetic Polypeptide
WSWSDGYYVYFDY

SEQ ID NO: 16 - protein, 122 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 17 - protein, 122 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 18 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 19 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 20 - protein, 505 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 530 aa, Artificial Sequence
  Synthetic Polypeptide
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 22 - protein, 993 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 511 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 24 - protein, 16 aa, Artificial Sequence
  Synthetic Polypeptide
RSSQSLVHSDGNTYLS

SEQ ID NO: 25 - protein, 7 aa, Artificial Sequence
  Synthetic Polypeptide
RISRRFS

SEQ ID NO: 26 - protein, 9 aa, Artificial Sequence
  Synthetic Polypeptide
MQSTHVPRT

SEQ ID NO: 27 - protein, 5 aa, Artificial Sequence
  Synthetic Polypeptide
NYGMH

SEQ ID NO: 28 - protein, 17 aa, Artificial Sequence
  Synthetic Polypeptide
VIWYDGSDKYYADSVRG

SEQ ID NO: 29 - protein, 15 aa, Artificial Sequence
  Synthetic Polypeptide
DGYDILTGNPRDFDY

SEQ ID NO: 30 - protein, 124 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 31 - protein, 112 aa, Artificial Sequence
  Synthetic Polypeptide
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 32 - protein, 251 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 33 - protein, 506 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 34 - protein, 5 aa, Artificial Sequence
  Synthetic Polypeptide
DYYMT

SEQ ID NO: 35 - protein, 17 aa, Artificial Sequence
  Synthetic Polypeptide
YISSSGSTIYYADSVKG

SEQ ID NO: 36 - protein, 8 aa, Artificial Sequence
  Synthetic Polypeptide
DRNSHFDY

SEQ ID NO: 37 - protein, 11 aa, Artificial Sequence
  Synthetic Polypeptide
RASQGINTWLA

SEQ ID NO: 38 - protein, 7 aa, Artificial Sequence
  Synthetic Polypeptide
GASGLQS

SEQ ID NO: 39 - protein, 9 aa, Artificial Sequence
  Synthetic Polypeptide
QQAKSFPRT

SEQ ID NO: 40 - protein, 117 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 41 - protein, 107 aa, Artificial Sequence
  Synthetic Polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 42 - protein, 239 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 43 - protein, 494 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 982 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 982 aa, Artificial Sequence; identical to SEQ ID NO: 44
  Synthetic Polypeptide

SEQ ID NO: 46 - protein, 5 aa, Artificial Sequence
  Synthetic Polypeptide
SYGMH

SEQ ID NO: 47 - protein, 17 aa, Artificial Sequence
  Synthetic Polypeptide
FIWYEGSNKYYAESVKD

SEQ ID NO: 48 - protein, 16 aa, Artificial Sequence
  Synthetic Polypeptide
RAGIIGTIGYYYGMDV

SEQ ID NO: 49 - protein, 11 aa, Artificial Sequence
  Synthetic Polypeptide
SGDRLGEKYTS

SEQ ID NO: 50 - protein, 7 aa, Artificial Sequence
  Synthetic Polypeptide
QDTKRPS

SEQ ID NO: 51 - protein, 9 aa, Artificial Sequence
  Synthetic Polypeptide
QAWESSTVV

SEQ ID NO: 52 - protein, 125 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 53 - protein, 107 aa, Artificial Sequence
  Synthetic Polypeptide
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 54 - protein, 247 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 55 - protein, 507 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 56 - protein, 7 aa, Artificial Sequence
  Synthetic Polypeptide
NARMGVS

SEQ ID NO: 57 - protein, 16 aa, Artificial Sequence
  Synthetic Polypeptide
HIFSNDEKSYSTSLKN

SEQ ID NO: 58 - protein, 14 aa, Artificial Sequence
  Synthetic Polypeptide
IVGYGSGWYGFFDY

SEQ ID NO: 59 - protein, 11 aa, Artificial Sequence
  Synthetic Polypeptide
RASQGIRNDLG

SEQ ID NO: 60 - protein, 7 aa, Artificial Sequence
  Synthetic Polypeptide
AASTLQS

SEQ ID NO: 61 - protein, 9 aa, Artificial Sequence
  Synthetic Polypeptide
LQHNSYPLT

SEQ ID NO: 62 - protein, 124 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 63 - protein, 107 aa, Artificial Sequence
  Synthetic Polypeptide
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 64 - protein, 246 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 65 - protein, 501 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 66 - protein, 989 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 5 aa, Artificial Sequence
  Synthetic Polypeptide
SYYWS

SEQ ID NO: 68 - protein, 16 aa, Artificial Sequence
  Synthetic Polypeptide
YVYYSGTTNYNPSLKS

SEQ ID NO: 69 - protein, 10 aa, Artificial Sequence
  Synthetic Polypeptide
IAVTGFYFDY

SEQ ID NO: 70 - protein, 12 aa, Artificial Sequence
  Synthetic Polypeptide
RASQRVNNNYLA

SEQ ID NO: 71 - protein, 7 aa, Artificial Sequence
  Synthetic Polypeptide
GASSRAT

SEQ ID NO: 72 - protein, 9 aa, Artificial Sequence
  Synthetic Polypeptide
QQYDRSPLT

SEQ ID NO: 73 - protein, 118 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 74 - protein, 108 aa, Artificial Sequence
  Synthetic Polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 75 - protein, 241 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 76 - protein, 496 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 77 - protein, 982 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 5 aa, Artificial Sequence
  Synthetic Polypeptide
SYGMH

SEQ ID NO: 79 - protein, 17 aa, Artificial Sequence
  Synthetic Polypeptide
VISYEGSNKYYAESVKG

SEQ ID NO: 80 - protein, 13 aa, Artificial Sequence
  Synthetic Polypeptide
DRGTIFGNYGLEV

SEQ ID NO: 81 - protein, 16 aa, Artificial Sequence
  Synthetic Polypeptide
RSSQSLLHKNAFNYLD

SEQ ID NO: 82 - protein, 7 aa, Artificial Sequence
  Synthetic Polypeptide
LGSNRAS

SEQ ID NO: 83 - protein, 9 aa, Artificial Sequence
  Synthetic Polypeptide
MQALQTPFT

SEQ ID NO: 84 - protein, 122 aa, Artificial Sequence
  Synthetic Polypeptide
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 85 - protein, 112 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 86 - protein, 503 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 87 - protein, 991 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 5 aa, Artificial Sequence
  Synthetic Polypeptide
NHIIH

SEQ ID NO: 89 - protein, 17 aa, Artificial Sequence
  Synthetic Polypeptide
YINPYPGYHAYNEKFQG

SEQ ID NO: 90 - protein, 12 aa, Artificial Sequence
  Synthetic Polypeptide
DGYYRDTDVLDY

SEQ ID NO: 91 - protein, 11 aa, Artificial Sequence
  Synthetic Polypeptide
QASQDISNYLN

SEQ ID NO: 92 - protein, 7 aa, Artificial Sequence
  Synthetic Polypeptide
YTSRLHT

SEQ ID NO: 93 - protein, 9 aa, Artificial Sequence
  Synthetic Polypeptide
QQGNTLPWT

SEQ ID NO: 94 - protein, 121 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 95 - protein, 107 aa, Artificial Sequence
  Synthetic Polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 96 - protein, 243 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 97 - protein, 986 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 504 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 99 - protein, 986 aa, Artificial Sequence; identical to SEQ ID NO: 97
  Synthetic Polypeptide

SEQ ID NO: 100 - protein, 5 aa, Artificial Sequence
  Synthetic Polypeptide
DYYMY

SEQ ID NO: 101 - protein, 17 aa, Artificial Sequence
  Synthetic Polypeptide
IISDAGYYTYYSDIIKG

SEQ ID NO: 102 - protein, 12 aa, Artificial Sequence
  Synthetic Polypeptide
GFPLLRHGAMDY

SEQ ID NO: 103 - protein, 11 aa, Artificial Sequence
  Synthetic Polypeptide
KASQNVDANVA

SEQ ID NO: 104 - protein, 7 aa, Artificial Sequence
  Synthetic Polypeptide
SASYVYW

SEQ ID NO: 105 - protein, 9 aa, Artificial Sequence
  Synthetic Polypeptide
QQYDQQLIT

SEQ ID NO: 106 - protein, 121 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 107 - protein, 107 aa, Artificial Sequence
  Synthetic Polypeptide
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 108 - protein, 243 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 109 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 986 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 984 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 5 aa, Artificial Sequence
  Synthetic Polypeptide
DYYMY

SEQ ID NO: 113 - protein, 17 aa, Artificial Sequence
  Synthetic Polypeptide
IISDGGYYTYYSDIIKG

SEQ ID NO: 114 - protein, 12 aa, Artificial Sequence
  Synthetic Polypeptide
GFPLLRHGAMDY

SEQ ID NO: 115 - protein, 11 aa, Artificial Sequence
  Synthetic Polypeptide
KASQNVDTNVA

SEQ ID NO: 116 - protein, 7 aa, Artificial Sequence
  Synthetic Polypeptide
SASYVYW

SEQ ID NO: 117 - protein, 9 aa, Artificial Sequence
  Synthetic Polypeptide
QQYDQQLIT

SEQ ID NO: 118 - protein, 121 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 119 - protein, 107 aa, Artificial Sequence
  Synthetic Polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 120 - protein, 243 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 121 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 122 - protein, 986 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 5 aa, Artificial Sequence
  Synthetic Polypeptide
TYAMS

SEQ ID NO: 124 - protein, 17 aa, Artificial Sequence
  Synthetic Polypeptide
AISGSGGRTFYAESVEG

SEQ ID NO: 125 - protein, 11 aa, Artificial Sequence
  Synthetic Polypeptide
HDYSNYPYFDY

SEQ ID NO: 126 - protein, 12 aa, Artificial Sequence
  Synthetic Polypeptide
RASQSVRSTYLA

SEQ ID NO: 127 - protein, 7 aa, Artificial Sequence
  Synthetic Polypeptide
GASSRAT

SEQ ID NO: 128 - protein, 9 aa, Artificial Sequence
  Synthetic Polypeptide
QQYGDLPFT

SEQ ID NO: 129 - protein, 120 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 130 - protein, 108 aa, Artificial Sequence
  Synthetic Polypeptide
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 131 - protein, 986 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 5 aa, Artificial Sequence
  Synthetic Polypeptide
GYYMH

SEQ ID NO: 133 - protein, 17 aa, Artificial Sequence
  Synthetic Polypeptide
WINPNSGGTKYAQKFQG

SEQ ID NO: 134 - protein, 16 aa, Artificial Sequence
  Synthetic Polypeptide
DRITVAGTYYYYGMDV

SEQ ID NO: 135 - protein, 11 aa, Artificial Sequence
  Synthetic Polypeptide
RASQGVNNWLA

SEQ ID NO: 136 - protein, 7 aa, Artificial Sequence
  Synthetic Polypeptide
TASSLQS

SEQ ID NO: 137 - protein, 9 aa, Artificial Sequence
  Synthetic Polypeptide
QQANSFPIT

SEQ ID NO: 138 - protein, 125 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 139 - protein, 107 aa, Artificial Sequence
  Synthetic Polypeptide
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 140 - protein, 247 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 141 - protein, 502 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 142 - protein, 990 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 125 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 144 - protein, 107 aa, Artificial Sequence; identical to SEQ ID NO: 139
  Synthetic Polypeptide

SEQ ID NO: 145 - protein, 247 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 146 - protein, 502 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 990 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 5 aa, Artificial Sequence
  Synthetic Polypeptide
NHGMH

SEQ ID NO: 149 - protein, 17 aa, Artificial Sequence
  Synthetic Polypeptide
GIWSEGSNKYYADAVKG

SEQ ID NO: 150 - protein, 12 aa, Artificial Sequence
  Synthetic Polypeptide
ATYTTGWSYFDY

SEQ ID NO: 151 - protein, 11 aa, Artificial Sequence
  Synthetic Polypeptide
SGDKLGDKYAS

SEQ ID NO: 152 - protein, 7 aa, Artificial Sequence
  Synthetic Polypeptide
QDAKRPS

SEQ ID NO: 153 - protein, 9 aa, Artificial Sequence
  Synthetic Polypeptide
QAFHQSTWV

SEQ ID NO: 154 - protein, 121 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSS

SEQ ID NO: 155 - protein, 106 aa, Artificial Sequence
  Synthetic Polypeptide
SYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVL

SEQ ID NO: 156 - protein, 985 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 5 aa, Artificial Sequence
  Synthetic Polypeptide
NHAMH

SEQ ID NO: 158 - protein, 17 aa, Artificial Sequence
  Synthetic Polypeptide
GIWSEGSNKYYAESVKG

SEQ ID NO: 159 - protein, 12 aa, Artificial Sequence
  Synthetic Polypeptide
ATYTTGWSYFDY

SEQ ID NO: 160 - protein, 11 aa, Artificial Sequence
  Synthetic Polypeptide
SGDKLGDKYAS

SEQ ID NO: 161 - protein, 7 aa, Artificial Sequence
  Synthetic Polypeptide
QDRKRPS

SEQ ID NO: 162 - protein, 9 aa, Artificial Sequence
  Synthetic Polypeptide
QAYDASTWV

SEQ ID NO: 163 - protein, 121 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSS

SEQ ID NO: 164 - protein, 106 aa, Artificial Sequence
  Synthetic Polypeptide
SYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVL

SEQ ID NO: 165 - protein, 985 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 5 aa, Artificial Sequence
  Synthetic Polypeptide
GYYWS

SEQ ID NO: 167 - protein, 16 aa, Artificial Sequence
  Synthetic Polypeptide
DIDASGSTKYNPSLKS

SEQ ID NO: 168 - protein, 12 aa, Artificial Sequence
  Synthetic Polypeptide
KKYSTVWSYFDN

SEQ ID NO: 169 - protein, 11 aa, Artificial Sequence
  Synthetic Polypeptide
SGDKLGDKYAS

SEQ ID NO: 170 - protein, 7 aa, Artificial Sequence
  Synthetic Polypeptide
QDRKRPS

SEQ ID NO: 171 - protein, 9 aa, Artificial Sequence
  Synthetic Polypeptide
QAWGSSTAV

SEQ ID NO: 172 - protein, 120 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 173 - protein, 106 aa, Artificial Sequence
  Synthetic Polypeptide
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 174 - protein, 984 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 5 aa, Artificial Sequence
  Synthetic Polypeptide
GYYWS

SEQ ID NO: 176 - protein, 16 aa, Artificial Sequence
  Synthetic Polypeptide
DIDYSGSTKYNPSLKS

SEQ ID NO: 177 - protein, 12 aa, Artificial Sequence
  Synthetic Polypeptide
KKYSTVWSYFDY

SEQ ID NO: 178 - protein, 11 aa, Artificial Sequence
  Synthetic Polypeptide
SGDKLGDKYAN

SEQ ID NO: 179 - protein, 7 aa, Artificial Sequence
  Synthetic Polypeptide
HDNKRPS

SEQ ID NO: 180 - protein, 9 aa, Artificial Sequence
  Synthetic Polypeptide
QAYGISSAV

SEQ ID NO: 181 - protein, 120 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSS

SEQ ID NO: 182 - protein, 106 aa, Artificial Sequence
  Synthetic Polypeptide
SYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVL

SEQ ID NO: 183 - protein, 984 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSSGGGGSGGGGSGGGGSSYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 989 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 987 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 501 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 187 - protein, 989 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 987 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 250 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 190 - protein, 250 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK